SEQUENCE LISTING (85 sequences)

SEQ ID NO: 1 - protein, 5 aa, Artificial Sequence
  Synthetic Sequence
  REPEAT (1)..(1): A spacer amino acid such as serine or glycine
  REPEAT (2)..(2): A positively charged amino acid such as arginine, histidine or lysine
  REPEAT (3)..(3): A spacer amino acid such as serine or glycine
  REPEAT (4)..(4): A positively charged amino acid such as arginine, histidine or lysine
  REPEAT (5)..(5): A spacer amino acid such as serine or glycine
XXXXX

SEQ ID NO: 2 - protein, 5 aa, Artificial Sequence
  Synthetic Sequence
  REPEAT (1)..(1): A spacer amino acid such as serine or glycine
  REPEAT (2)..(2): A negatively charged amino acid such as aspartic acid or glutamic acid
  REPEAT (3)..(3): A spacer amino acid such as serine or glycine
  REPEAT (4)..(4): A negatively charged amino acid such as aspartic acid or glutamic acid
  REPEAT (5)..(5): A spacer amino acid such as serine or glycine
XXXXX

SEQ ID NO: 3 - protein, 10 aa, Artificial Sequence
  Synthetic Sequence
  MISC_FEATURE (1)..(1): A spacer amino acid such as serine or glycine
  REPEAT (2)..(2): A spacer amino acid such as serine or glycine
  MISC_FEATURE (3)..(3): A positively charged amino acid such as arginine, histidine or lysine
  REPEAT (4)..(4): A positively charged amino acid such as arginine, histidine or lysine
  MISC_FEATURE (5)..(5): A spacer amino acid such as serine or glycine
  REPEAT (6)..(6): A spacer amino acid such as serine or glycine
  MISC_FEATURE (7)..(7): A positively charged amino acid such as arginine, histidine or lysine
  REPEAT (8)..(8): A positively charged amino acid such as arginine, histidine or lysine
  MISC_FEATURE (9)..(9): A spacer amino acid such as serine or glycine
  REPEAT (10)..(10): A spacer amino acid such as serine or glycine
XXXXXXXXXX

SEQ ID NO: 4 - protein, 10 aa, Artificial Sequence
  Synthetic Sequence
  MISC_FEATURE (1)..(1): A spacer amino acid such as serine or glycine
  REPEAT (2)..(2): A spacer amino acid such as serine or glycine
  MISC_FEATURE (3)..(3): A negatively charged amino acid such as aspartic acid or glutamic acid
  REPEAT (4)..(4): A negatively charged amino acid such as aspartic acid or glutamic acid
  MISC_FEATURE (5)..(5): A spacer amino acid such as serine or glycine
  REPEAT (6)..(6): A spacer amino acid such as serine or glycine
  MISC_FEATURE (7)..(7): A negatively charged amino acid such as aspartic acid or glutamic acid
  REPEAT (8)..(8): A negatively charged amino acid such as aspartic acid or glutamic acid
  MISC_FEATURE (9)..(9): A spacer amino acid such as serine or glycine
  REPEAT (10)..(10): A spacer amino acid such as serine or glycine
XXXXXXXXXX

SEQ ID NO: 5 - protein, 6 aa, Artificial Sequence
  Synthetic Sequence
  REPEAT (1)..(1): A spacer amino acid such as serine or glycine
  REPEAT (2)..(2): A positively charged amino acid such as arginine, histidine or lysine
  REPEAT (4)..(4): A spacer amino acid such as serine or glycine
  REPEAT (5)..(5): A positively charged amino acid such as arginine, histidine or lysine
  REPEAT (6)..(6): A spacer amino acid such as serine or glycine
XXCXXX

SEQ ID NO: 6 - protein, 6 aa, Artificial Sequence
  Synthetic Sequence
  REPEAT (1)..(1): A spacer amino acid such as serine or glycine
  REPEAT (2)..(2): A negatively charged amino acid such as aspartic acid or glutamic acid
  REPEAT (4)..(4): A spacer amino acid such as serine or glycine
  REPEAT (5)..(5): A negatively charged amino acid such as aspartic acid or glutamic acid
  REPEAT (6)..(6): A spacer amino acid such as serine or glycine
XXCXXX

SEQ ID NO: 7 - protein, 12 aa, Artificial Sequence
  Synthetic Sequence
GSGSRKGGKRGS

SEQ ID NO: 8 - protein, 12 aa, Artificial Sequence
  Synthetic Sequence
GSGSRKCGKRGS

SEQ ID NO: 9 - protein, 12 aa, Artificial Sequence
  Synthetic Sequence
GSGSDEGGEDGS

SEQ ID NO: 10 - protein, 12 aa, Artificial Sequence
  Synthetic Sequence
GSGSDECGEDGS

SEQ ID NO: 11 - protein, 6 aa, Artificial Sequence
  Synthetic polymer.
RKGGKR

SEQ ID NO: 12 - protein, 12 aa, Artificial Sequence
  Synthetic polymer.
GSGSRKGGKRGS

SEQ ID NO: 13 - protein, 6 aa, Artificial Sequence
  Synthetic polymer.
DEGGED

SEQ ID NO: 14 - protein, 12 aa, Artificial Sequence
  Synthetic polymer.
GSGSDEGGEDGS

SEQ ID NO: 15 - protein, 234 aa, Artificial Sequence
  Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 16 - protein, 222 aa, Artificial Sequence
  Synthetic polymer.
CPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 17 - protein, 232 aa, Artificial Sequence
  Synthetic polymer.
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 232 aa, Artificial Sequence
  Synthetic polymer.
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 19 - protein, 227 aa, Artificial Sequence
  Synthetic polymer.
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 20 - protein, 249 aa, Artificial Sequence
  Synthetic polymer.
GSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGS

SEQ ID NO: 21 - protein, 249 aa, Artificial Sequence
  Synthetic polymer.
GSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGS

SEQ ID NO: 22 - protein, 222 aa, Artificial Sequence; identical to SEQ ID NO: 16
  Synthetic polymer.

SEQ ID NO: 23 - skipped

SEQ ID NO: 24 - protein, 220 aa, Artificial Sequence
  Synthetic polymer.
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCA

SEQ ID NO: 25 - protein, 113 aa, Artificial Sequence
  Synthetic polymer.
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLV

SEQ ID NO: 26 - protein, 225 aa, Artificial Sequence
  Synthetic polymer.
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAG

SEQ ID NO: 27 - protein, 115 aa, Artificial Sequence
  Synthetic polymer.
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVA

SEQ ID NO: 28 - protein, 223 aa, Artificial Sequence
  Synthetic polymer.
EVSWFSVKGPAEPITVLLGTEATLPCQLSPEQSAARMHIRWYRAQPTPAVLVFHNGQEQGEVQMPEYRGRTQMVRQAIDMGSVALQIQQVQASDDGLYHCQFTDGFTSQEVSMELRVIGLGSAPLVHMTGPENDGIRVLCSSSGWFPKPKVQWRDTSGNMLLSSSELQTQDREGLFQVEVSLLVTDRAIGNVICSIQNPMYDQEKSKAILLPEPFFPKTCPWK

SEQ ID NO: 29 - protein, 163 aa, Artificial Sequence
  Synthetic polymer.
EQLPEYSQRTSLVKEQFHQGTAAVRILNVQAPDSGIYICHFKQGVFYEEAILELKVAAMGSVPEVYIKGPEDGGVCVVCITSGWYPEPQVHWKDSRGEKLTASLEIHSEDAQGLFRTETSLVVRDSSVRNVTCSTFNPILGQEKAMAMFLPEPFFPKVSPWKP

SEQ ID NO: 30 - protein, 220 aa, Artificial Sequence
  Synthetic polymer.
QWQVTGPGKFVQALVGEDAVFSCSLFPETSAEAMEVRFFRNQFHAVVHLYRDGEDWESKQMPQYRGRTEFVKDSIAGGRVSLRLKNITPSDIGLYGCWFSSQIYDEEATWELRVAALGSLPLISIVGYVDGGIQLLCLSSGWFPQPTAKWKGPQGQDLSSDSRANADGYSLYDVEISIIVQENAGSILCSIHLAEQSHEVESKVLIGETFFQPSPWRLAS

SEQ ID NO: 31 - protein, 219 aa, Artificial Sequence
  Synthetic polymer.
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPW

SEQ ID NO: 32 - protein, 221 aa, Artificial Sequence
  Synthetic polymer.
QWQVFGPDKPVQALVGEDAAFSCFLSPKTNAEAMEVRFFRGQFSSVVHLYRDGKDQPFMQMPQYQGRTKLVKDSIAEGRISLRLENITVLDAGLYGCRISSQSYYQKAIWELQVSALGSVPLISITGYVDRDIQLLCQSSGWFPRPTAKWKGPQGQDLSTDSRTNRDMHGLFDVEISLTVQENAGSISCSMRHAHLSREVESRVQIGDTFFEPISWHLATK

SEQ ID NO: 33 - protein, 216 aa, Artificial Sequence
  Synthetic polymer.
APFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPR

SEQ ID NO: 34 - protein, 215 aa, Artificial Sequence
  Synthetic polymer.
APFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLP

SEQ ID NO: 35 - protein, 432 aa, Artificial Sequence
  Synthetic polymer.
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 36 - protein, 220 aa, Artificial Sequence; identical to SEQ ID NO: 24
  Synthetic polymer.

SEQ ID NO: 37 - protein, 233 aa, Artificial Sequence
  Synthetic polymer.
QFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWM

SEQ ID NO: 38 - protein, 219 aa, Artificial Sequence
  Synthetic polymer.
QVTVVGPTDPILAMVGENTTLRCCLSPEENAEDMEVRWFQSQFSPAVFVYKGGRERTEEQKEEYRGRTTFVSKDSRGSVALIIHNVTAEDNGIYQCYFQEGRSCNEAILHLVVAGLDSEPVIEMRDHEDGGIQLECISGGWYPKPLTVWRDPYGEVVPALKEVSTPDADSLFMVTTAVIIRDKSVRNVSCSINDTLLGQKKESVIFIPESFMPSRSPCV

SEQ ID NO: 39 - protein, 220 aa, Artificial Sequence; identical to SEQ ID NO: 30
  Synthetic polymer.

SEQ ID NO: 40 - protein, 225 aa, Artificial Sequence; identical to SEQ ID NO: 26
  Synthetic polymer.

SEQ ID NO: 41 - protein, 219 aa, Artificial Sequence; identical to SEQ ID NO: 31
  Synthetic polymer.

SEQ ID NO: 42 - protein, 219 aa, Artificial Sequence
  Synthetic polymer.
QFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPW

SEQ ID NO: 43 - protein, 221 aa, Artificial Sequence; identical to SEQ ID NO: 32
  Synthetic polymer.

SEQ ID NO: 44 - protein, 222 aa, Artificial Sequence
  Synthetic polymer.
SSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGASAWK

SEQ ID NO: 45 - protein, 228 aa, Artificial Sequence
  Synthetic polymer.
SIWKADFDVTGPHAPILAMAGGHVELQCQLFPNISAEDMELRWYRCQPSLAVHMHERGMDMDGEQKWQYRGRTTFMSDHVARGKAMVRSHRVTTFDNRTYCCRFKDGVKFGEATVQVQVAGLGREPRIQVTDQQDGVRAECTSAGCFPKSWVERRDFRGQARPAVTNLSASATTRLWAVASSLTLWDRAVEGLSCSISSPLLPERRKVAESHLPATFSRSSQFTAWKA

SEQ ID NO: 46 - protein, 14 aa, Artificial Sequence
  Synthetic polymer.
ALWEPGYSSDWIKT

SEQ ID NO: 47 - protein, 13 aa, Artificial Sequence
  Synthetic polymer.
ALWEDVGDYYKKL

SEQ ID NO: 48 - protein, 12 aa, Artificial Sequence
  Synthetic polymer.
ALWEFESFYKKL

SEQ ID NO: 49 - protein, 13 aa, Artificial Sequence
  Synthetic polymer.
ATWDGWAFYYKKL

SEQ ID NO: 50 - protein, 6 aa, Artificial Sequence
  Synthetic polymer.
APHKKL

SEQ ID NO: 51 - protein, 12 aa, Artificial Sequence
  Synthetic polymer.
ATWSPTLYYKKL

SEQ ID NO: 52 - protein, 10 aa, Artificial Sequence
  Synthetic polymer.
ATWDVERKKL

SEQ ID NO: 53 - protein, 13 aa, Artificial Sequence
  Synthetic polymer.
ATWDGISSDWIKT

SEQ ID NO: 54 - protein, 13 aa, Artificial Sequence
  Synthetic polymer.
ATWDGPKNYYKKL

SEQ ID NO: 55 - protein, 11 aa, Artificial Sequence
  Synthetic polymer.
ATWDGQYYKKL

SEQ ID NO: 56 - protein, 11 aa, Artificial Sequence
  Synthetic polymer.
ATWDANYYKKL

SEQ ID NO: 57 - protein, 14 aa, Artificial Sequence
  Synthetic polymer.
AIYPGPGPSGWFKI

SEQ ID NO: 58 - protein, 12 aa, Artificial Sequence
  Synthetic polymer.
ATWDYSTGWFKI

SEQ ID NO: 59 - protein, 10 aa, Artificial Sequence
  Synthetic polymer.
ATWDGRDKKL

SEQ ID NO: 60 - protein, 15 aa, Artificial Sequence
  Synthetic polymer.
ALWEEPQELGKKIKV

SEQ ID NO: 61 - protein, 10 aa, Artificial Sequence
  Synthetic polymer.
ALWFFRWFKI

SEQ ID NO: 62 - protein, 14 aa, Artificial Sequence
  Synthetic polymer.
ALWEVQELGKKIKV

SEQ ID NO: 63 - protein, 17 aa, Artificial Sequence
  Synthetic polymer.
ALWETTFWGELGKKIKV

SEQ ID NO: 64 - protein, 10 aa, Artificial Sequence
  Synthetic polymer.
ALWEPQDKKL

SEQ ID NO: 65 - protein, 14 aa, Artificial Sequence
  Synthetic polymer.
ALWEPQELGKKIKV

SEQ ID NO: 66 - protein, 19 aa, Artificial Sequence
  Synthetic polymer.
AMRAFTFPRYWGWDTDKLI

SEQ ID NO: 67 - protein, 21 aa, Artificial Sequence
  Synthetic polymer.
AYRSLLTLPWMGDTPYTDKLI

SEQ ID NO: 68 - protein, 9 aa, Artificial Sequence
  Synthetic polymer.
ALGGFGRLI

SEQ ID NO: 69 - protein, 22 aa, Artificial Sequence
  Synthetic polymer.
ALGELTTPLSLPWGILNTDKLI

SEQ ID NO: 70 - protein, 17 aa, Artificial Sequence
  Synthetic polymer.
ALGELPPFLGWSTDKLI

SEQ ID NO: 71 - protein, 23 aa, Artificial Sequence
  Synthetic polymer.
ALVGPFLFLPGGYTVPTDTDKLI

SEQ ID NO: 72 - protein, 18 aa, Artificial Sequence
  Synthetic polymer.
ALGVTIFLPWGGHTDKLI

SEQ ID NO: 73 - protein, 21 aa, Artificial Sequence
  Synthetic polymer.
ALGELSNFLRGTGIGETDKLI

SEQ ID NO: 74 - protein, 19 aa, Artificial Sequence
  Synthetic polymer.
ALGDPLILPWGIRDTDKLI

SEQ ID NO: 75 - protein, 15 aa, Artificial Sequence
  Synthetic polymer.
AATAPTLSGGSDKLI

SEQ ID NO: 76 - protein, 27 aa, Artificial Sequence
  Synthetic polymer.
ALGELLVPTYWGIQFSYSHPPYTDKLI

SEQ ID NO: 77 - protein, 13 aa, Artificial Sequence
  Synthetic polymer.
AFGYTGEYTDKLI

SEQ ID NO: 78 - protein, 22 aa, Artificial Sequence
  Synthetic polymer.
ALGENKWGLPSLVLGIRSDKLI

SEQ ID NO: 79 - protein, 16 aa, Artificial Sequence
  Synthetic polymer.
ALGELLWPSYAGDKLI

SEQ ID NO: 80 - protein, 27 aa, Artificial Sequence
  Synthetic polymer.
TLGELLVPTYWGIQFSYSHPPYTDKLI

SEQ ID NO: 81 - protein, 27 aa, Artificial Sequence
  Synthetic polymer.
TLGELLVPACWGIQFSYSHPPYTDKLI

SEQ ID NO: 82 - protein, 22 aa, Artificial Sequence
  Synthetic polymer.
DLGENKWGLPSLVLGIRSDKLI

SEQ ID NO: 83 - protein, 22 aa, Artificial Sequence
  Synthetic polymer.
DLEENKWGLPSLVLGIRSDKLI

SEQ ID NO: 84 - protein, 27 aa, Artificial Sequence
  Synthetic polymer.
ALGKLLGPTYWGIQFSYSHPPYTDKLI

SEQ ID NO: 85 - protein, 27 aa, Artificial Sequence
  Synthetic polymer.
ALGELLVPTYWGTQFSYSHPPYTDKLI

(19) United States
(12) Patent Application Publication
(10) Pub. No.: US 2023/0408523 A1
(43) Pub. Date: Dec. 21, 2023

(54) METHODS OF IDENTIFYING GAMMA DELTA T CELL-MODULATING AGENTS

(71) Applicant: Shattuck Labs, Inc., Austin, TX (US)

(72) Inventors: George FROMM, Austin, TX (US); Suresh DE SILVA, Austin, TX (US); Taylor SCHREIBER, Austin, TX (US)

(21) Appl. No.: 18/037,628

(22) PCT Filed: Nov. 18, 2021
(86) PCT No.: PCT/US21/59852

§ 371 Date: May 18, 2023

Related U.S. Application Data
(60) Provisional application No. 63/173,063, filed on Apr. 9, 2021.
(60) Provisional application No. 63/116,058, filed on Nov. 19, 2020.

(51) Int. Cl. G01N 33/574; G01N 33/569; C07K 14/705

(52) U.S. Cl. CPC G01N 33/57484; G01N 33/56972; C07K 14/70503; G01N 2333/7051; G01N 2333/70503; C07K 2319/33

(57) ABSTRACT

The present invention relates to, in part, methods that are useful for cancer treatment, and methods for selecting personalized treatment regimens.

### PRIORITY

This application claims the benefit of, and priority to, U.S. Provisional Application Nos. 63/116,058, filed Nov. 19, 2020; and 63/173,063, filed Apr. 9, 2021, the contents of which are hereby incorporated by reference in its entirety.

### TECHNICAL FIELD

The current disclosure relates to treatment of cancer.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SHK-037PC_116981-5037_ST25; date created: Nov. 11, 2021; file size: 77,920 bytes).

### BACKGROUND

Cancer is a significant health problem worldwide. Despite recent advances no universally successful method for prevention or treatment is currently available. Current therapies, which are generally based on a combination of chemotherapy or surgery and radiation, continue to prove inadequate in many patients.

A major limitation of current treatments for cancer is the selection of appropriate active agents for a patient.

It is common that sub-optimal chemotherapy is provided to a patient, resulting in unsuccessful treatment, including death, disease progression, unnecessary toxicity, and higher health care costs. Further, checkpoint inhibitors, while promising, are not effective in many patients.

Therefore, there remains a need for methods that are useful for evaluating cancer and related diseases and developing new therapies. The present disclosure fulfills these needs and further provides other related advantages.

### SUMMARY

Accordingly, the present invention provides, in part, methods for selecting patients for cancer treatment, and methods for cancer treatment, based on, for instance, abundance of certain gamma/delta T cell (for example, asjudged by the expression of specific gamma/delta T cell receptors (TCRs) and/or expression of butyrophilin family of proteins.

In one aspect, the present disclosure relates to a method for identifying a cancer therapy, the method comprising: (a) obtaining a biological sample, the sample comprising one or more gamma/delta T cells; (b) evaluating the sample for the presence, absence, or level of one or more gamma/delta T-cell receptor (TCR) chains; and (c) selecting a cancer therapy having an ability to signal at the gamma/delta TCR, the cancer therapy comprising one or more butyrophilin proteins, or a fragment thereof. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In one aspect, the present disclosure relates to a method for selecting a patient for a cancer treatment, the method comprising: (a) obtaining a biological sample, the sample comprising one or more gamma/delta T cells; (b) evaluating the sample for the presence, absence, or level of one or more gamma/delta T-cell receptor (TCR) chains; and (C) selecting a cancer therapy having an ability to signal at the gamma/delta TCR, the cancer therapy comprising one or more butyrophilin proteins, or a fragment thereof. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In one aspect, the present disclosure relates to a method for making an agent for the treatment of a cancer in a cancer patient, comprising: (a) obtaining the agent for the treatment of a cancer, the obtaining comprising: (i) evaluating the biological sample from a cancer patient for the presence, absence, or level of one or more gamma/delta T-cell receptor (TCR) chains; and (ii) selecting a cancer therapy having an ability to signal at the one or more gamma/delta TCR, the cancer therapy comprising one or more butyrophilin proteins, or a fragment thereof; (b) formulating the identified agent for administration to a cancer patient. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In one aspect, the present disclosure relates to a method for identifying a cancer therapy for a tumor, the method comprising: (a) obtaining a biological sample, the sample comprising one or more gamma/delta T cells; (b) evaluating the sample for the presence, absence, or level of one or more butyrophilin proteins; and (c) selecting a cancer therapy having an ability to signal at a gamma/delta TCR, the cancer therapy comprising the one or more butyrophilin proteins, or a fragment thereof. In embodiments, the evaluating comprises measuring the presence, absence, or level of one or more butyrophilin proteins selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the evaluating comprises measuring the presence, absence, or level of one or more butyrophilin proteins selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In one aspect, the present disclosure relates to a method for selecting a patient for a cancer treatment, the method comprising: (a) obtaining a biological sample, the sample comprising one or more gamma/delta T cells; (b) evaluating the sample for the presence, absence, or level of one or more butyrophilin proteins; and (c) selecting a cancer therapy having an ability to signal at a gamma/delta TCR, the cancer therapy comprising the one or more butyrophilin proteins, or a fragment thereof. In embodiments, the evaluating comprises measuring the presence, absence, or level of one or more butyrophilin proteins selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the evaluating comprises measuring the presence, absence, or level of one or more butyrophilin proteins selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In one aspect, the present disclosure relates to a method for making an agent for the treatment of a cancer in a cancer patient, comprising: (a) obtaining the agent for the treatment of a cancer, the obtaining comprising: (i) evaluating the biological sample from a cancer patient for the presence, absence, or level of one or more butyrophilin proteins: and (ii) selecting a cancer therapy having an ability to signal at a gamma/delta TCR, the cancer therapy comprising the one or more butyrophilin proteins, or a fragment thereof; (b) formulating the identified agent for administration to a cancer patient. In embodiments, the evaluating comprises measuring the presence, absence, or level of one or more gamma/delta T-cell receptor (TR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In embodiments, the biological sample may be a tissue sample. For example, the tissue sample may be selected from fresh tissue sample, frozen tumor tissue specimen, cultured cells, circulating tumor cells, and a formalin-fixed paraffin-embedded tumor tissue specimen. In embodiments, the tissue sample is a biopsy sample. In embodiments, the biopsy sample is selected from endoscopic biopsy, bone marrow biopsy, endoscopic biopsy (e.g. cystoscopy, bronchoscopy and colonoscopy), needle biopsy (e.g. fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g. shave biopsy, punch biopsy, and incisional biopsy) and surgical biopsy. In embodiments, the tissue sample is selected from bone, bone marrow, lung, brain, liver, adrenal gland, colon, intestine, esophagus, pancreas, urinary bladder, breast, lymph node, and skin. In embodiments, the biological sample comprises a body fluid.

In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample is a tumor sample. In embodiments, the tumor is metastatic. In embodiments, the tumor has metastasized to a tissue or an organ. In embodiments, the tissue or the organ is selected from bone, bone marrow, lung, brain, liver, adrenal gland, colon, intestine, esophagus, pancreas, urinary bladder, breast, lymph node, and skin.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof.

In embodiments, the evaluating is performed by contacting the sample with an agent that specifically binds to one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, VδG, Vδ7, and Vδ8. In embodiments, the agent that specifically binds to one or more TCR is an antibody or fragment thereof. In embodiments, the antibody is a recombinant antibody, a monoclonal antibody, a polyclonal antibody, or fragment thereof.

In embodiments, the evaluating is performed by contacting the sample with an agent that specifically binds to one or more butyrophilin. In embodiments, the agent that specifically binds to one or more butyrophilin is an antibody or fragment thereof. In embodiments, the antibody is a recombinant antibody, a monoclonal antibody, a polyclonal antibody, or fragment thereof. In embodiments, the evaluating is performed by contacting the sample with an agent that specifically binds to one or more butyrophilin selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In embodiments, the level and/or activity of one or more butyrophilin is measured by contacting the sample with an agent that specifically binds to one or more of the nucleic acids. In embodiments, the agent that specifically binds to one or more of the nucleic acids is a nucleic acid primer or probe.

In embodiments, the level and/or activity of one or more TCR is measured by contacting the sample with an agent that specifically binds to one or more of the nucleic acids. In embodiments, the agent that specifically binds to one or more of the nucleic acids is a nucleic acid primer or probe.

In embodiments, when presence, or higher level in tumor compared to surrounding healthy tissue of: TCR Vγ9δ2 is detected, the cancer therapy comprising human BTN2A1, BTN3A1, BTN2A2 and/or BTN2A3 butyrophilin protein, or a fragment thereof is selected; and/or TCR Vδ4 is detected, the cancer therapy comprising human BTNL3 butyrophilin protein, or a fragment thereof is selected.

In embodiments, when presence, or higher level in tumor compared to surrounding healthy tissue of a butyrophilin protein is detected, the cancer therapy comprising the butyrophilin protein, or a fragment thereof is selected. For example, human BTN1A1 is detected, the cancer therapy comprising human BTN1A1 butyrophilin protein, or a fragment thereof is selected; human BTNL2 is detected, the cancer therapy comprising human BTNL2 butyrophilin protein, or a fragment thereof is selected; human BTN2A1 is detected, the cancer therapy comprising human BTN2A1 butyrophilin protein, or a fragment thereof is selected.

In embodiments, the cancer therapy comprises a heterodimeric protein comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the first domain comprises two of the same butyrophilin family proteins. In embodiments, the first domain comprises two different butyrophilin family proteins. In embodiments, the butyrophilin family proteins, or a fragment thereof comprise an Ig-like V-type domain. In embodiments, the butyrophilin family proteins, or a fragment thereof are derived from native full length proteins.

In embodiments, the cancer therapy comprises a heterodimeric protein comprising an alpha chain and a beta chain wherein the alpha chain and the beta chain each independently comprise (a) a first domain comprising a butyrophilin family protein, or fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the first domain comprises two of the same butyrophilin family proteins. In embodiments, the first domain comprises two different butyrophilin family proteins. In embodiments, the butyrophilin family proteins, or a fragment thereof comprise an Ig-like V-type domain. In embodiments, the butyrophilin family proteins, or a fragment thereof are derived from native full length proteins. In embodiments, the first domain comprises a polypeptide having an amino acid sequence of: (a) any one of SEQ ID NOs: 24 to 45, or a fragment thereof; and (b) any one of SEQ ID NOs: 24 to 45, or a fragment thereof. In embodiments, the linker comprises a polypeptide having an amino acid sequence of one of more of SEQ ID NOs: 1-14. In embodiments, the linker comprises a polypeptide having an amino acid sequence of SEQ ID NOs: 15-22.

In embodiments, the targeting domain is an antibody, or antigen binding fragment thereof. In embodiments, the targeting domain is an antibody-like molecule, or antigen binding fragment thereof. In embodiments, the antibody-like molecule is an scFv molecule. In embodiments, the targeting domain is an extracellular domain.

In embodiments, the targeting domain is capable of binding an antigen on the surface of a cancer cell. In embodiments, the targeting domain specifically binds CD19. In embodiments, the targeting domain specifically binds PSMA. In embodiments, the targeting domain specifically binds CD33. In embodiments, the targeting domain specifically binds CD20. In embodiments, the targeting domain specifically binds CLL-1.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence.

In embodiments, the first and/or second charge polarized core domain further comprise peptides having positively and/or negatively charged amino acid residues at the amino and/or carboxy terminus of the charge polarized core domain.

In embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ7δ4. In embodiments, the modulation of a gamma delta T cell is activation of a gamma delta T cell. In embodiments, the heterodimeric protein is capable of forming a synapse between a gamma delta T cell and a tumor cell and/or the heterodimeric protein is capable of contemporaneous activation and targeting of gamma delta T cells to tumor cells.

### BRIEF DESCRIPTION OF THE FIGURES

FIG. 1A to FIG. 1C show the gamma and delta chain pairing in matched peripheral blood and dissociated tumor tissue from cancer patients. FIG. 1A shows a schematic representation of the method used for single cell sequencing from a prostate cancer patient to determine gamma and delta chain pairing in peripheral blood mononuclear cells (PBMCs, FIG. 1B) and dissociated tumor tissue (FIG. 1C). Briefly, PBMCs and dissociated tumor tissue were obtained from a prostate adenocarcinoma patient. Gamma delta T cells in the samples were single cell sorted into 96 well plates by flow cytometry following antibody staining of the gamma delta TCR. RNA was prepared and the gamma and delta chains were sequenced using the iPAIR PCR technique (iRepertoire). The iPair analyzer software was used to identify gamma and delta chain sequences and pairings on a single cell level. FIG. 1B is a graph showing gamma and delta chain pairings in the patient's PBMCs. FIG. 1C is a graph showing gamma and delta chain pairings in the patient's dissociated tumor samples.

FIG. 2 shows a schematic representation of the method used for single cell sequencing from cancer patients to determine gamma and delta chain pairing in tumor tissue and tumor infiltrating lymphocytes (TIL). Briefly, freshly resected tumor tissue (colorectal cancer is shown in the illustration) was mechanically dissociated to create a single cell suspension of tumor and tumor infiltrating lymphocytes (TIL). Gamma delta T cells in the samples were stained with a pan γδ TCR antibody and single cell sorted into 96 well plates by flow cytometry. The RNA was extracted from the cells and TCRγ and TCRδ chains were amplified and sequenced using the iPAIR PCR technique (iRepertoire). The iPair analyzer software was used to identify gamma and delta chain sequences and pairings on a single cell level.

FIG. 3A and FIG. 3B show the CDR3 tree map comparison of gamma chain usage in tumor vs PBMC. FIG. 3A shows the diversity of the gamma chain CDR3 sequences in dissociated tumor. The sequences of the top 10 CDR3 are listed below the CDR3 tree map. FIG. 3B shows the diversity of the gamma chain CDR3 sequences in PBMCs. The sequences of the top 10 CDR3 are listed below the CDR3 tree map.

FIG. 4A and FIG. 4B show the CDR3 tree map comparison of delta chain usage in tumor vs PBMC. FIG. 4A shows the diversity of the delta chain CDR3 sequences in dissociated tumor. The sequences of the top 10 CDR3 are listed below the CDR3 tree map. FIG. 4B shows the diversity of the delta chain CDR3 sequences in PBMCs. The sequences of the top 10 CDR3 are listed below the CDR3 tree map.

FIG. 5A to FIG. 5D show the γδ TCR (TRGV and TRDV) compositions in the select cancer genome atlas (TCGA) tumor types. The γδ TCR (TRGV and TRDV) compositions for colorectal adenocarcinoma (FIG. 5A), diffuse large B cell lymphoma (FIG. 5B), sarcoma (FIG. 5C), and stomach adenocarcinoma (FIG. 5D) are shown.

FIG. 6A to FIG. 6F show CDR3 sequence analysis of the γδ TCR in peripheral blood and the tumor. FIG. 6A shows the single cell RNA sequencing of Vγ chain from colorectal tumors. FIG. 6B shows the single cell RNA sequencing of Vδ chain from colorectal tumors. FIG. 6C shows the single cell RNA sequencing of Vγ chain from matched peripheral blood. FIG. 6D shows the single cell RNA sequencing of Vδ chain from matched peripheral blood. FIG. 6E shows the CDR3 sequence analysis of the Vγ and Vδ chains in colorectal cancer tumor. FIG. 6F shows the CDR3 sequence analysis of the Vγ and Vδ chains in peripheral blood.

FIG. 7A to FIG. 7F show the correlation of BTN/L and γδ TCR expression in colorectal adenocarcinoma. Spearman correlation analysis was performed on the RNA expression of select BTN/L proteins (BTN3A1 (FIG. 7A), BTN2A1 (FIG. 7B), BTNL3 (FIG. 7C) and BTNL8 (FIG. 7D), gamma chain (TRGV9) (FIG. 7E) and delta chain (TRDV1) (FIG. 7F) detected in colorectal adenocarcinoma tumor through the analysis of TCGA datasets.

FIG. 8A and FIG. 8B show the development of a Jurkat-76 cell line-based in vitro assay to screen BTN/L pairs that activate specific γδ TCRs detected using single cell RNA sequencing. FIG. 8A shows a schematic representation of the assay. Shown is an illustration of development of Jurkat-76 reporter cell lines (top), which exhibits CD69 expression upon γδ T cell activation (bottom). FIG. 8B shows the representative data from one of the Jurkat-76 reporter systems (expressing Vγ9δ2 TCR).

FIG. 9 shows a non-limiting schematic of the generation of GADLEN therapeutics. Without being bound by theory, the GADLEN therapeutics activate of the γδ TCR through the BTN/L heterodimer domains and cause targeted killing of tumor cells.

FIG. 10A to FIG. 10C show the gamma (TRGV) and delta (TRDV) chain T-cell receptor usage in melanoma tumors (FIG. 10A), non-small cell lung tumors (FIG. 10B) and colorectal cancer tumors (FIG. 10C).

### DETAILED DESCRIPTION

The current disclosure is based, in part, on the discovery that tissue-specific preferences for individual gamma/delta T cell TCRs, with corresponding tissue-specific preferences for individual butyrophilin proteins in human cancer patients, including melanoma, prostate and colon cancer patients, and that, the specific gamma/delta T cell populations are preferentially activated by specific butyrophilin heterodimers in a lock-and-key fashion. Accordingly, the present disclosure relates to selecting patients for anticancer therapy with chimeric proteins that have the ability to, inter alia, target the tissue-specific gamma delta T cells and cause their activation, while also forming a synapse with the tumor cells.

A major mechanism of acquired resistance to immune checkpoint inhibition involves downregulation of antigen presentation, including the major histocompatibility complex (MHC I) complex itself. Downregulation of antigen presentation on MHC I can render tumor cells invisible to alpha/beta (αβ) T cell directed therapies. Gamma/delta (γδ) T cells are a small subset of the overall T cell compartment but are characterized by increased cytolytic capacity relative to αβ T cells. Rather than via MHC I, γδ T cells recognize target cells via a complex of heterodimerized butyrophilin (BTN) proteins. Thus, display of BTN heterodimers on the surface of tumor cells may enhance immunity to tumors that have downregulated MHC I, or which express low abundance or low affinity antigens.

The data presented herein, inter alia, identified tissue-specific enrichment of individual γδ T cell TCRs, with corresponding tissue-specific preferences for individual BTN proteins. Based on this information, a panel of distinct heterodimeric BTN proteins were generated. The data showed, inter alia, that the specific γδ T cell populations are preferentially activated by specific BTN heterodimers, without being bound by theory, in a lock-and-key fashion. Accordingly, in aspects, the present disclosure relates to a method for identifying a cancer therapy, the method comprising: (a) obtaining a biological sample, the sample comprising one or more gamma/delta T cells; (b) evaluating the sample for the presence, absence, or level of one or more gamma/delta T-cell receptor (TCR) chains and/or one or more butyrophilin proteins; and (c) selecting a cancer therapy having an ability to signal at the gamma/delta TCR, the one or more butyrophilin proteins, or a fragment thereof.

The Heterodimeric Proteins Suitable for the Cancer Therapy

In one aspect, the present disclosure relates to cancer therapy with a heterodimeric protein comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the heterodimeric protein comprises two polypeptide chains, wherein the first polypeptide chain and the second polypeptide chain comprise (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the heterodimeric protein comprises two individual polypeptide chains which self-associate. In embodiments, the first domain comprising one or more butyrophilin family proteins, or a fragment thereof of the first polypeptide chain and the second polypeptide chain are the same. In embodiments, the second domain comprising a targeting domain of the first and the second polypeptide chain are the same. In embodiments, the linker that adjoins the first and second domain are the same.

In one aspect, the present disclosure relates to cancer therapy with a heterodimeric protein comprising an alpha chain and a beta chain, wherein the alpha chain and the beta chain each independently comprise: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the heterodimeric protein comprises the alpha chain and the beta chain, which self-associate. In embodiments, the first domain comprising one or more butyrophilin family proteins, or a fragment thereof of the alpha chain and the beta chain are the same. In embodiments, the second domain comprising a targeting domain of the alpha chain and the beta chain are the same. In embodiments, the linker that adjoins the first and second domain of the alpha chain and the beta chain are the same.

Exemplary heterodimeric proteins suitable for use in the methods disclosed herein are disclosed in PCT International Patent Application Publication No. WO2020146393, and PCT International Patent Application No. PCT/US2021/027294, the entire contents of which are hereby incorporated by reference.

The Butyrophilin Family Proteins, or Fragments Thereof

The heterodimeric proteins suitable for cancer therapy disclosed herein comprise a first domain comprising one or more butyrophilin family proteins, or a fragment thereof. In embodiments, the butyrophilin family proteins are selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the first domain comprises: (a) any one of BTN1A1, BTN2A1, BTN2A2, BTN2A3, BTN3A1, BTN3A2, BTN3A3, BTNL2, BTNL3, BTNL8, BTNL9, BTNL10, and SKINTL; and (b) any one of BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the first domain comprises: (a) any one of human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL, and (b) any one of human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In embodiments, the first domain comprises a fragment of butyrophilin family proteins, wherein the fragment is capable of binding a gamma delta T cell receptor and is optionally an extracellular domain, optionally comprising one or more of an immunoglobulin V (IgV)- and IgC-like domain. In embodiments, the first domain comprises a fragment of butyrophilin family proteins, wherein the fragment is capable of binding a gamma delta T cell receptor selected from a Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In embodiments, the first domain comprises two of the same butyrophilin family proteins. In embodiments, wherein the first domain comprises two different butyrophilin family proteins. In embodiments, the butyrophilin family proteins comprise a V-type domain. Suitable butyrophilin family proteins or fragments thereof are derived from the native butyrophilin family proteins that comprise a B30.2 domain in the cytosolic tail of the full length protein.

In embodiments, the first domain is a portion of Butyrophilin subfamily 2 member A1 (BTN2A1). In embodiments, the first domain comprises substantially all the extracellular domain of BTN2A1. In embodiments, the first domain is capable of binding a gamma delta T cell receptor (e.g. Vγ9δ2). BTN2A1 is also known as BT2.1, BTF1. In embodiments, the portion of BTN2A1 is a portion of the extracellular domain of BTN2A1. In embodiments, the present chimeric protein further comprises a domain, e.g., the extracellular domain BTN2A1.

The amino acid sequence of extracellular domain of human BTN2A1, which is an illustrative amino acid sequence of human BTN2A1 suitable in the current disclosure is the following:

(SEQ ID NO: 24)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLVVAGLGSK
PLISMRGHEDGGIRLECISRGWYPKPLTVWRDPYGGVAPA
LKEVSMPDADGLFMVTTAVIIRDKSVRNMSCSINNTLLGQ
KKESVIFIPESFMPSVSPCA

In some embodiments, the fragment of extracellular domain of human BTN2A1, suitable in the current disclosure is the Ig-like V-type domain, which has the following sequence:

(SEQ ID NO: 25)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLV

In embodiments, the heterodimeric protein suitable for cancer therapy comprises the extracellular domain of human BTN2A1 which has the amino acid sequence of SEQ ID NO: 24 or SEQ ID NO: 25. In embodiments, the present chimeric proteins may comprise the extracellular domain of BTN2A1 as described herein, or a variant or functional fragment thereof. For instance, the chimeric protein may comprise a sequence of the extracellular domain of BTN2A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN2A1 as described herein.

BTN2A1 derivatives can be constructed from available structural data, including a homology model described by Karunakaran et al., Butyrophilin-2A1 Directly Binds Germline-Encoded Regions of the Vγ9Vδ2 TCR and Is Essential for Phosphoantigen Sensing, Immunity. 52(3): 487-498 (2020). Moreover, without wishing to be bound by theory, the protein structure homology-model of BTN2A1 is available at SWISS-MODEL repository. Bienert et al., “The SWISS-MODEL Repository—new features and functionality.” Nucleic Acids Research, 45(D1): D313-D319 (2017). Additional structural insight obtained from mutagenesis. Rigau et al., Butyrophilin 2A1 is essential for phosphoantigen reactivity by γδ T cells. Science 367(6478):eaay5516 (2020).

In embodiments, the first domain is a portion of Butyrophilin subfamily 3 member A1 (BTN3A1). In embodiments, the first domain comprises substantially all the extracellular domain of BTN3A1. In embodiments, the first domain is capable of binding a gamma delta T cell receptor (e.g. Vγ9δ2). BTN3A1 is also known as BTF5. In embodiments, the portion of BTN3A1 is a portion of the extracellular domain of BTN3A1. In embodiments, the present chimeric protein further comprises a domain, e.g., the extracellular domain BTN3A1.

The amino acid sequence of extracellular domain of human BTN3A1, which is an illustrative amino acid sequence of human BTN3A1 suitable in the current disclosure is the following:

(SEQ ID NO: 26)
QFSVLGPSGPILAMVGEDADLPCHLFPTMSAETMELKWVS
SSLRQVVNVYADGKEVEDRQSAPYRGRTSILRDGITAGKA
ALRIHNVTASDSGKYLCYFQDGDFYEKALVELKVAALGSD
LHVDVKGYKDGGIHLECRSTGWYPQPQIQWSNNKGENIPT
VEAPVVADGVGLYAVAASVIMRGSSGEGVSCTIRSSLLGL
EKTASISIADPFFRSAQRWIAALAG

In some embodiments, the fragment of extracellular domain of human BTN3A1, is the Ig-like V-type 1 domain, which has the following sequence:

(SEQ ID NO: 27)
QFSVLGPSGPILAMVGEDADLPCHLFPTMSAETMELKWVS
SSLRQVVNVYADGKEVEDRQSAPYRGRTSILRDGITAGKA
ALRIHNVTASDSGKYLCYFQDGDFYEKALVELKVA

In embodiments, the heterodimeric protein suitable for cancer therapy comprises the extracellular domain of human BTN3A1 which has the amino acid sequence of SEQ ID NO: 26 or SEQ ID NO: 27. In embodiments, the present chimeric proteins may comprise the extracellular domain of BTN3A1 as described herein, or a variant or functional fragment thereof. For instance, the chimeric protein may comprise a sequence of the extracellular domain of BTN3A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN3A1 as described herein.

BTN3A1 derivatives can be constructed from available structural data, including the following: Palakodeti et al., The molecular basis for modulation of human V(gamma)9V(delta)2 T cell responses by CD277/Butyrophilin-3 (BTN3A)-specific antibodies, J Biol Chem 287: 32780-32790 (2012); Vavassori et al., Butyrophilin 3A1 binds phosphorylated antigens and stimulates human gamma delta T cells. Nat Immunol 14: 908-916 (2013); Sandstrom et al., The Intracellular B30.2 Domain of Butyrophilin 3A1 Binds Phosphoantigens to Mediate Activation of Human V gamma 9V delta 2 T Cells. Immunity 40: 490-500 (2014); Rhodes et al., Activation of Human Gammadelta T Cells by Cytosolic Interactions of Btn3A1 with Soluble Phosphoantigens and the Cytoskeletal Adaptor Periplakin. J Immunol 194: 2390 (2015); Gu et al., Phosphoantigen-induced conformational change of butyrophilin 3A1 (BTN3A1) and its implication on V gamma 9V delta 2 T cell activation. Proc Natl Acad Sci USA 114: E7311-E7320 (2017); Salim et al., BTN3A1 Discriminates gamma delta T Cell Phosphoantigens from Nonantigenic Small Molecules via a Conformational Sensor in Its B30.2 Domain. ACS Chem Biol 12: 2631-2643 (2017); Yang et al., A Structural Change in Butyrophilin upon Phosphoantigen Binding Underlies Phosphoantigen-Mediated V gamma 9V delta 2 T Cell Activation. Immunity 50: 1043 (2019).

In embodiments, the first domain comprises a portion of BTN2A1. In embodiments, the portion of BTN2A1 is an extracellular domain of BTN2A1, or a γδ T-cell receptor (e.g. γ9δ2)-binding fragment thereof.

In embodiments, the first domain comprises a portion of BTN3A1. In embodiments, the portion of BTN3A1 is an extracellular domain of BTN3A1, or a γδ T-cell receptor (e.g. γ9δ2)-binding fragment thereof.

An illustrative amino acid sequence of mouse BTNL1 suitable in the present disclosure is:

(SEQ ID NO: 28)
EVSWFSVKGPAEPITVLLGTEATLPCQLSPEQSAARMHIRWYRAQPTPA
VLVFHNGQEQGEVQMPEYRGRTQMVRQAIDMGSVALQIQQVQASDDGLY
HCQFTDGFTSQEVSMELRVIGLGSAPLVHMTGPENDGIRVLCSSSGWFP
KPKVQWRDTSGNMLLSSSELQTQDREGLFQVEVSLLVTDRAIGNVICSI
QNPMYDQEKSKAILLPEPFFPKTCPWK

An illustrative amino acid sequence of mouse BTNL6 suitable in the present disclosure:

(SEQ ID NO: 29)
EQLPEYSQRTSLVKEQFHQGTAAVRILNVQAPDSGIYICHFKQGVFYEE
AILELKVAAMGSVPEVYIKGPEDGGVCVVCITSGWYPEPQVHWKDSRGE
KLTASLEIHSEDAQGLFRTETSLVVRDSSVRNVTCSTFNPILGQEKAMA
MFLPEPFFPKVSPWKP

An illustrative amino acid sequence of human BTNL3 suitable in the present disclosure is the following:

(SEQ ID NO: 30)
QWQVTGPGKFVQALVGEDAVFSCSLFPETSAEAMEVRFFRNQFHAVVHL
YRDGEDWESKQMPQYRGRTEFVKDSIAGGRVSLRLKNITPSDIGLYGCW
FSSQIYDEEATWELRVAALGSLPLISIVGYVDGGIQLLCLSSGWFPQPT
AKWKGPQGQDLSSDSRANADGYSLYDVEISIIVQENAGSILCSIHLAEQ
SHEVESKVLIGETFFQPSPWRLAS

An illustrative amino acid sequence of human BTN3A2 suitable in the present disclosure

(SEQ ID NO: 31)
QFSVLGPSGPILAMVGEDADLPCHLFPTMSAETMELKWVSSSLRQVVNV
YADGKEVEDRQSAPYRGRTSILRDGITAGKAALRIHNVTASDSGKYLCY
FQDGDFYEKALVELKVAALGSNLHVEVKGYEDGGIHLECRSTGWYPQPQ
IQWSNAKGENIPAVEAPVVADGVGLYEVAASVIMRGGSGEGVSCIIRNS
LLGLEKTASISIADPFFRSAQPW

An illustrative amino acid sequence of human BTNL8 suitable in the present disclosure is as follows:

(SEQ ID NO: 32)
QWQVFGPDKPVQALVGEDAAFSCFLSPKTNAEAMEVRFFRGQFSSVVHL
YRDGKDQPFMQMPQYQGRTKLVKDSIAEGRISLRLENITVLDAGLYGCR
ISSQSYYQKAIWELQVSALGSVPLISITGYVDRDIQLLCQSSGWFPRPT
AKWKGPQGQDLSTDSRTNRDMHGLFDVEISLTVQENAGSISCSMRHAHL
SREVESRVQIGDTFFEPISWHLATK

In embodiments, the heterodimeric protein suitable for cancer therapy may comprise one or more of the extracellular domain of human BTN2A1, BTN3A1, BTNL1, BTNL3, BTN3A2, BTNL8, which have the amino acid sequence of SEQ ID NO: 24, SEQ ID NO: 26, SEQ ID NO: 28, SEQ ID NO: 30, SEQ ID NO: 31, and SEQ ID NO: 32, respectively. In embodiments, the present chimeric proteins may comprise the extracellular domain of one or more of the extracellular domain of human BTN2A1, BTN3A1, BTNL1, BTNL3, BTN3A2, BTNL8 as described herein, or a variant or functional fragment thereof. In embodiments, the fragment is an Ig-like V-type domain from one or more of SEQ ID NO: 24, SEQ ID NO: 26, SEQ ID NO: 28, SEQ ID NO: 30, SEQ ID NO: 31, and SEQ ID NO: 32 For instance, the chimeric protein may comprise a sequence of the extracellular domain of BTN3A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of one or more of the extracellular domain of human BTN2A1, BTN3A1, BTNL1, BTNL3, BTN3A2, BTNL8 as described herein.

In various embodiments, the present heterodimeric proteins comprise two independent binding domains, each from one subunit of a heterodimeric human protein. Illustrative proteins that may be formed as part of the heterodimeric protein of the invention are provided in the table below. In various embodiments, the present heterodimeric proteins have one of the illustrative proteins provided in the table below. In various embodiments, the present heterodimeric proteins have two of the illustrative proteins provided in the table below.

Additional illustrative butyrophilin-like (BTNL) family protein which may be incorporated into the present compositions and methods include the following proteins (as used herein, “Entry” refers to the protein entry in the Uniprot database and “Entry name” refers to the protein entry in the Uniprot database):

| | | | SEQ |
|---|---|---|---|
| Entry/ | Protein names | | ID |
| Name | Gene names | ECD Sequence | NO |
| Q13410 | Butyrophilin | APFDVIGPPEPILAVVGEDAELPCRLSPNASAEHLELRWFR | 33 |
| BT1A1_HUMAN | subfamily 1 | KKVSPAVLVHRDGREQEAEQMPEYRGRATLVQDGIAKGR | |
| | member A1 | VALRIRGVRVSDDGEYTCFFREDGSYEEALVHLKVAALGS | |
| | Butyrophilin | DPHISMQVQENGEICLECTSVGWYPEPQVQWRTSKGEKF | |
| | subfamily 1 | PSTSESRNPDEEGLFTVAASVIIRDTSAKNVSCYIQNLLLG | |
| | member A1; | QEKKVEISIPASSLPR | |
| | BTN1A1 BTN | | |
| Q13410 | Butyrophilin | APFDVIGPPEPILAVVGEDAELPCRLSPNASAEHLELRWFR | 34 |
| BT1A1_HUMAN | subfamily 1 | KKVSPAVLVHRDGREQEAEQMPEYRGRATLVQDGIAKGR | |
| | member A1 | VALRIRGVRVSDDGEYTCFFREDGSYEEALVHLKVAALGS | |
| | BTN1A1 BTN | DPHISMQVQENGEICLECTSVGWYPEPQVQWRTSKGEKF | |
| | | PSTSESRNPDEEGLFTVAASVIIRDTSAKNVSCYIQNLLLG | |
| | | QEKKVEISIPASSLP | |
| Q4VAN1Q4VAN1_ | BTN1A1 protein | | |
| HUMAN | BTN1A1 | | |
| Q4VAN2 | Butyrophilin, | | |
| Q4VAN2_HUMAN | subfamily 1, | | |
| | member A. | | |
| | BTN1A1 | | |
| Q9UIR0 | Butyrophilin- | KQSEDFRVIGPAHPILAGVGEDALLTCQLLPKRTTMHVEVR | 35 |
| BTNL2_HUMAN | like | WYRSEPSTPVFVHRDGVEVTEMQMEEYRGWVEWIENGI | |
| | protein 2 | AKGNVALKIHNIQPSDNGQYWCHFQDGNYCGETSLLLKVA | |
| | BTNL2 | GLGSAPSIHMEGPGESGVQLVCTARGWFPEPQVYWEDIR | |
| | | GEKLLAVSEHRIQDKDGLFYAEATLVVRNASAESVSCLVHN | |
| | | PVLTEEKGSVISLPEKLQTELASLKVNGPSQPILVRVGEDIQ | |
| | | LTCYLSPKANAQSMEVRWDRSHRYPAVHVYMDGDHVAG | |
| | | EQMAEYRGRTVLVSDAIDEGRLTLQILSARPSDDGQYRCL | |
| | | FEKDDVYQEASLDLKVVSLGSSPLITVEGQEDGEMQPMC | |
| | | SSDGWFPQPHVPWRDMEGKTIPSSSQALTQGSHGLFHV | |
| | | QTLLRVTNISAVDVTCSISIPFLGEEKIATFSLSGW | |
| F8WBA1 | Butyrophilin- | | |
| F8WBA1_HUMAN | like | | |
| | protein 2 | | |
| | BTNL2 | | |
| F6UPS5 | Butyrophilin- | | |
| F6UPS5_HUMAN | like | | |
| | protein 2 | | |
| | BTNL2 | | |
| F8WDK6 | Butyrophilin- | | |
| F8WDK6_HUMAN | like | | |
| | protein 2 | | |
| | BTNL2 | | |
| A0A0G2JJ84 | Butyrophilin- | | |
| A0A0G2JJ84_ | like | | |
| HUMAN | protein 2 | | |
| | BTNL2 | | |
| X5D146 | BTNL2 | | |
| X5D146_HUMAN | BTNL2 | | |
| A0A0G2JPB7 | BTNL2 | | |
| A0A0G2JPB7_ | | | |
| HUMAN | | | |
| A0PJV4 | BTNL2 protein | | |
| A0PJV4_HUMAN | BTNL2 | | |
| I7HPB5 | Butyrophilin- | | |
| I7HPB5_HUMAN | like | | |
| | 2 (MHC class | | |
| | II a . . . | | |
| | BTNL2 RP5- | | |
| | 1077I5.2-002 | | |
| A0A1U9X7B7 | BTNL2 | | |
| A0A1U9X7B7_ | | | |
| HUMAN | | | |
| X5CF33 | BTNL2 | | |
| X5CF33_HUMAN | BTNL2 hCG_43715 | | |
| A0A1U9X7C0 | BTNL2 | | |
| A0A1U9X7C0_ | | | |
| HUMAN | | | |
| A0A1U9X7C3 | Truncated | | |
| A0A1U9X7C3_ | BTNL2 | | |
| HUMAN | | | |
| A0A1U9X7C4 | Truncated | | |
| A0A1U9X7C4_ | BTNL2 | | |
| HUMAN | | | |
| Q7KYR7 | Butyrophilin | QFIVVGPTDPILATVGENTTLRCHLSPEKNAEDMEVRWFR | 36 |
| BT2A1_HUMAN | subfamily 2 | SQFSPAVFVYKGGRERTEEQMEEYRGRTTFVSKDISRGS | |
| | member A1 | VALVIHNITAQENGTYRCYFQEGRSYDEAILHLVVAGLGSK | |
| | BTN2A1 BT2.1, | PLISMRGHEDGGIRLECISRGWYPKPLTVWRDPYGGVAPA | |
| | BTF1 | LKEVSMPDADGLFMVTTAVIIRDKSVRNMSCSINNTLLGQK | |
| | | KESVIFIPESFMPSVSPCA | |
| H7BYC3 | Butyrophilin | | |
| H7BYC3_HUMAN | subfamily 2 | | |
| | member A1 | | |
| | BTN2A1 | | |
| H7C542 | Butyrophilin | | |
| H7C542_HUMAN | subfamily 2 | | |
| | member A1 | | |
| | BTN2A1 | | |
| C9JNC3 | Butyrophilin | | |
| C9JNC3_HUMAN | subfamily 2 | | |
| | member A1 | | |
| | BTN2A1 | | |
| Q8WVV5 | Butyrophilin | QFTVVGPANPILAMVGENTTLRCHLSPEKNAEDMEVRWF | 37 |
| BT2A2_HUMAN | subfamily 2 | RSQFSPAVFVYKGGRERTEEQMEEYRGRITFVSKDINRGS | |
| | member A2 | VALVIHNVTAQENGIYRCYFQEGRSYDEAILRLVVAGLGSK | |
| | BTN2A2 BT2.2, | PLIEIKAQEDGSIWLECISGGWYPEPLTVWRDPYGEVVPAL | |
| | BTF2 | KEVSIADADGLFMVTTAVIIRDKYVRNVSCSVNNTLLGQEK | |
| | | ETVIFIPESFMPSASPWMVALAVILTASPWM | |
| A0A024R038 | Butyrophilin, | | |
| A0A024R038_ | subfamily 2, | | |
| HUMAN | member A . . . | | |
| | BTN2A2 | | |
| | hCG_1980289 | | |
| C9J8J5 | Butyrophilin | | |
| C9J8J5_HUMAN | subfamily 2 | | |
| | member A2 | | |
| | BTN2A2 | | |
| C9IZY2 | Butyrophilin | | |
| C9IZY2_HUMAN | subfamily 2 | | |
| | member A2 | | |
| | BTN2A2 | | |
| B4E3J1 | cDNA | | |
| B4E3J1_HUMAN | FLJ52852, | | |
| | highly similar | | |
| | to Ho . . . | | |
| C9IY66 | Butyrophilin | | |
| C9IY66_HUMAN | subfamily 2 | | |
| | member A2 | | |
| | BTN2A2 | | |
| C9J8R3 | Butyrophilin | | |
| C9J8R3_HUMAN | subfamily 2 | | |
| | member A2 | | |
| | BTN2A2 | | |
| C9JAJ6 | Butyrophilin | | |
| C9JAJ6_HUMAN | subfamily 2 | | |
| | member A2 | | |
| | BTN2A2 | | |
| C9JWH2 | Butyrophilin | | |
| C9JWH2_HUMAN | subfamily 2 | | |
| | member A2 | | |
| | BTN2A2 | | |
| H7C4E8 | Butyrophilin | | |
| H7C4E8_HUMAN | subfamily 2 | | |
| | member A2 | | |
| | BTN2A2 | | |
| F8WC65 | Butyrophilin | | |
| F8WC65_HUMAN | subfamily 2 | | |
| | member A2 | | |
| | BTN2A2 | | |
| Q96KV6 | Putative | QVTVVGPTDPILAMVGENTTLRCCLSPEENAEDMEVRWF | 38 |
| BT2A3_HUMAN | butyrophilin | QSQFSPAVFVYKGGRERTEEQKEEYRGRTTFVSKDSRGS | |
| | subfamily 2 | VALIIHNVTAEDNGIYQCYFQEGRSCNEAILHLVVAGLDSEP | |
| | m . . . | VIEMRDHEDGGIQLECISGGWYPKPLTVWRDPYGEVVPAL | |
| | BTN2A3P | KEVSTPDADSLFMVTTAVIIRDKSVRNVSCSINDTLLGQKK | |
| | BTN2A3 | ESVIFIPESFMPSRSPCV | |
| Q6UXE8 | Butyrophilin- | QWQVTGPGKFVQALVGEDAVFSCSLFPETSAEAMEVRFF | 39 |
| BTNL3_HUMAN | like | RNQFHAVVHLYRDGEDWESKQMPQYRGRTEFVKDSIAG | |
| | protein 3 | GRVSLRLKNITPSDIGLYGCWFSSQIYDEEATWELRVAALG | |
| | BTNL3 BTNLR, | SLPLISIVGYVDGGIQLLCLSSGWFPQPTAKWKGPQGQDL | |
| | COLF4100, | SSDSRANADGYSLYDVEISIIVQENAGSILCSIHLAEQSHEV | |
| | UNQ744/ | ESKVLIGETFFQPSPWRLAS | |
| | PRO1472 | | |
| L8EAU7 | Alternative | | |
| L8EAU7_HUMAN | protein BTNL3 | | |
| | BTNL3 | | |
| O00481 | Butyrophilin | QFSVLGPSGPILAMVGEDADLPCHLFPTMSAETMELKWV | 40 |
| BT3A1_HUMAN | subfamily 3 | SSSLRQVVNVYADGKEVEDRQSAPYRGRTSILRDGITAGK | |
| | member A1 | AALRIHNVTASDSGKYLCYFQDGDFYEKALVELKVAALGS | |
| | BTN3A1 BTF5 | DLHVDVKGYKDGGIHLECRSTGWYPQPQIQWSNNKGENI | |
| | | PTVEAPVVADGVGLYAVAASVIMRGSSGEGVSCTIRSSLLG | |
| | | LEKTASISIADPFFRSAQRWIAALAG | |
| E7EPR2 | Butyrophilin | | |
| E7EPR2_HUMAN | subfamily 3 | | |
| | member A1 | | |
| | BTN3A1 | | |
| E9PFB8 | Butyrophilin | | |
| E9PFB8_HUMAN | subfamily 3 | | |
| | member A1 | | |
| | BTN3A1 | | |
| A6PVC0 | Butyrophilin | | |
| A6PVC0_HUMAN | subfamily 3 | | |
| | member A1 | | |
| | BTN3A1 | | |
| P78410 | Butyrophilin | QFSVLGPSGPILAMVGEDADLPCHLFPTMSAETMELKWV | 41 |
| BT3A2_HUMAN | subfamily 3 | SSSLRQVVNVYADGKEVEDRQSAPYRGRTSILRDGITAGK | |
| | member A2 | AALRIHNVTASDSGKYLCYFQDGDFYEKALVELKVAALGS | |
| | BTN3A2 BT3.2, | NLHVEVKGYEDGGIHLECRSTGWYPQPQIQWSNAKGENI | |
| | BTF3, BTF4 | PAVEAPVVADGVGLYEVAASVIMRGGSGEGVSCIIRNSLLG | |
| | | LEKTASISIADPFFRSAQPW | |
| A0A024QZZ1 | Butyrophilin, | | |
| A0A024QZZ1_ | subfamily 3, | | |
| HUMAN | member A . . . | | |
| | BTN3A2 | | |
| | hCG_17993 | | |
| S4R3N0 | Butyrophilin | | |
| S4R3N0_HUMAN | subfamily 3 | | |
| | member A2 | | |
| | BTN3A2 | | |
| E9PJE9 | Butyrophilin | | |
| E9PJE9_HUMAN | subfamily 3 | | |
| | member A2 | | |
| | BTN3A2 | | |
| E9PIU5 | Butyrophilin | | |
| E9PIU5_HUMAN | subfamily 3 | | |
| | member A2 | | |
| | BTN3A2 | | |
| E9PRR1 | Butyrophilin | | |
| E9PRR1_HUMAN | subfamily 3 | | |
| | member A2 | | |
| | BTN3A2 | | |
| E9PRX1 | Butyrophilin | | |
| E9PRX1_HUMAN | subfamily 3 | | |
| | member A2 | | |
| | BTN3A2 | | |
| O00478 | Butyrophilin | QFSVLGPSGPILAMVGEDADLPCHLFPTMSAETMELRWV | 42 |
| BT3A3_HUMAN | subfamily 3 | SSSLRQVVNVYADGKEVEDRQSAPYRGRTSILRDGITAGK | |
| | member A3 | AALRIHNVTASDSGKYLCYFQDGDFYEKALVELKVAALGS | |
| | BTN3A3 BTF3 | DLHIEVKGYEDGGIHLECRSTGWYPQPQIKWSDTKGENIP | |
| | | AVEAPVVADGVGLYAVAASVIMRGSSGGGVSCIIRNSLLGL | |
| | | EKTASISIADPFFRSAQPW | |
| A0A024R042 | Butyrophilin, | | |
| A0A024R042_ | subfamily 3, | | |
| HUMAN | member A . . . | | |
| | BTN3A3 | | |
| | hCG_17992 | | |
| A0A089GIA6 | Butyrophilin | | |
| A0A089GIA6_ | subfamily 3 | | |
| HUMAN | member A3 . . . | | |
| | BTN3A3 | | |
| C9JUV8 | Butyrophilin | | |
| C9JUV8_HUMAN | subfamily 3 | | |
| | member A3 | | |
| | BTN3A3 | | |
| C9JQT8 | Butyrophilin | | |
| C9JQT8_HUMAN | subfamily 3 | | |
| | member A3 | | |
| | BTN3A3 | | |
| C9JVU4 | Butyrophilin | | |
| C9JVU4_HUMAN | subfamily 3 | | |
| | member A3 | | |
| | BTN3A3 | | |
| C9J3Q8 | Butyrophilin | | |
| C9J3Q8_HUMAN | subfamily 3 | | |
| | member A3 | | |
| | BTN3A3 | | |
| C9JZT5 | Butyrophilin | | |
| C9JZT5_HUMAN | subfamily 3 | | |
| | member A3 | | |
| | BTN3A3 | | |
| C9J877 | Butyrophilin | | |
| C9J877_HUMAN | subfamily 3 | | |
| | member A3 | | |
| | BTN3A3 | | |
| C9JNZ3 | Butyrophilin | | |
| C9JNZ3_HUMAN | subfamily 3 | | |
| | member A3 | | |
| | BTN3A3 | | |
| Q6UX41 | Butyrophilin- | QWQVFGPDKPVQALVGEDAAFSCFLSPKTNAEAMEVRFF | 43 |
| BTNL8_HUMAN | like | RGQFSSVVHLYRDGKDQPFMQMPQYQGRTKLVKDSIAEG | |
| | protein 8 | RISLRLENITVLDAGLYGCRISSQSYYQKAIWELQVSALGS | |
| | BTNL8 UNQ702/ | VPLISITGYVDRDIQLLCQSSGWFPRPTAKWKGPQGQDLS | |
| | PRO1347 | TDSRTNRDMHGLFDVEISLTVQENAGSISCSMRHAHLSRE | |
| | | VESRVQIGDTFFEPISWHLATK | |
| D6RIR7 | Butyrophilin- | | |
| D6RIR7_HUMAN | like | | |
| | protein 8 | | |
| | BTNL8 | | |
| D6R9I8 | Butyrophilin- | | |
| D6R9I8_HUMAN | like | | |
| | protein 8 | | |
| | BTNL8 | | |
| Q6UXG8 | Butyrophilin- | SSEVKVLGPEYPILALVGEEVEFPCHLWPQLDAQQMEIRW | 44 |
| BTNL9_HUMAN | like | FRSQTFNVVHLYQEQQELPGRQMPAFRNRTKLVKDDIAYG | |
| | protein 9 | SVVLQLHSIIPSDKGTYGCRFHSDNFSGEALWELEVAGLG | |
| | BTNL9 UNQ1900/ | SDPHLSLEGFKEGGIQLRLRSSGWYPKPKVQWRDHQGQ | |
| | PRO4346 | CLPPEFEAIVWDAQDLFSLETSVVVRAGALSNVSVSIQNLL | |
| | | LSQKKELVVQIADVFVPGASAWK | |
| A0A1S5UZ21 | Butyrophilin- | | |
| A0A1S5UZ21_ | like | | |
| HUMAN | protein 9 | | |
| | BTNL9 | | |
| B7Z4Y8 | Butyrophilin- | | |
| B7Z4Y8_HUMAN | like | | |
| | protein 9 | | |
| | BTNL9 | | |
| Q8N324 | BTNL9 protein | | |
| Q8N324_HUMAN | BTNL9 | | |
| A8MVZ5 | Butyrophilin- | SIWKADFDVTGPHAPILAMAGGHVELQCQLFPNISAEDME | 45 |
| BTNLA_HUMAN | like | LRWYRCQPSLAVHMHERGMDMDGEQKWQYRGRTTFMS | |
| | protein 10 | DHVARGKAMVRSHRVTTFDNRTYCCRFKDGVKFGEATVQ | |
| | BTNL10 | VQVAGLGREPRIQVTDQQDGVRAECTSAGCFPKSWVER | |
| | | RDFRGQARPAVTNLSASATTRLWAVASSLTLWDRAVEGLS | |
| | | CSISSPLLPERRKVAESHLPATFSRSSQFTAWKA | |

In embodiments, the heterodimeric protein suitable for cancer therapy may comprise one or more of the extracellular domain present in the amino acid of SEQ ID NO: 24, SEQ ID NO: 26, and SEQ ID NO: 28 to SEQ ID NO: 45. In embodiments, the present chimeric proteins may comprise the extracellular domain present in the amino acid of SEQ ID NO: 24, SEQ ID NO: 26, and SEQ ID NO: 28 to SEQ ID NO: 45 as described herein, or a variant or functional fragment thereof. In embodiments, the fragment is an Ig-like V-type domain from one or more of SEQ ID NO: 24, SEQ ID NO: 26, and SEQ ID NO: 28 to SEQ ID NO: 45. For instance, the chimeric protein may comprise a sequence of the extracellular domain of BTN3A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of present in the amino acid of SEQ ID NO: 24, SEQ ID NO: 26, and SEQ ID NO: 28 to 45 as described herein, and or one or more Ig-like V-type domain present therein.

The Second Domain Comprising a Targeting Domain

The heterodimers proteins of any of the embodiments disclosed herein comprise a second domain comprising a targeting domain. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv. In embodiments, the targeting domain is an extracellular domain. In embodiments, the targeting domain is capable of binding an antigen on the surface of a cancer cell. In embodiments, the targeting domain specifically binds a protein selected from CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDRNEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, CDH18, EPHA3, NY-eso-2, MAGE-A10, MAGE-A3, MAGE-A7, HER3, and GD3. In embodiments, the targeting domain comprises a portion of the extracellular domain of LAG-3, PD-1, TIGIT, CD19, or PSMA. In embodiments, the targeting domain specifically binds CD19. In embodiments, the targeting domain specifically binds PSMA. In embodiments, the targeting domain specifically binds CD33. In embodiments, the targeting domain specifically binds CD20. In embodiments, the targeting domain specifically binds CLL-1.

The Linker Domain that Adjoins the First and the Second Domain

In embodiments, the linker that adjoins the first and second domain comprises a charge polarized core domain. In various embodiments, each of the first and second charge polarized core domains comprises proteins having positively or negatively charged amino acid residues at the amino and carboxy terminus of the core domain. In an illustrative embodiment, the first charge polarized core domain may comprise a protein having positively charged amino acids at the amino terminus which are adjoined by alinker (e.g., a stabilizing domain) to a protein having negatively charged amino acid residues at the carboxy terminus. The second charge polarized core domain may comprise a protein having negatively charged amino acids at the amino terminus which are adjoined by a linker (e.g., a stabilizing domain) to a protein having positively charged amino acid residues at the carboxy terminus.

In another illustrative embodiment, the first charge polarized core domain may comprise a protein having negatively charged amino acids at the amino terminus which are adjoined by a linker (e.g., a stabilizing domain) to a protein having positively charged amino acid residues at the carboxy terminus. The second charge polarized core domain may comprise proteins having positively charged amino acids at the amino terminus which are adjoined by a linker (e.g., a stabilizing domain) to a protein having negatively charged amino acid residues at the carboxy terminus.

In various embodiments, formation of heterodimeric proteins is driven by electrostatic interactions between the positively charged and negatively charged amino acid residues located at the amino and carboxy termini of the first and second charge polarized core domains. Further, formation of homodimeric proteins is prevented by the repulsion between the positively charged amino acid residues or negatively charged amino acid residues located at the amino and carboxy termini of the first and second charge polarized core domains.

In various embodiments, the protein comprising positively and/or negatively charged amino acid residues at the amino or carboxy terminus of the charge polarized core domains is about 2 to about 50 amino acids long. For example, the protein comprising positively and/or negatively charged amino acid residues at either terminus of the charge polarized core domain may be about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the protein comprising positively charged amino acid residues may include one or more of amino acids selected from His, Lys, and Arg. In various embodiments, the protein comprising negatively charged amino acid residues may include one or more amino acids selected from Asp and Glu.

In various embodiments, each of the first and/or second charge polarized core domains may comprise a protein comprising an amino acid sequence as provided in the Table below or an amino acid sequence having at least 90%, or 93%, or 95%, or 97%, or 98%, or 99% identity thereto.

| SEQ ID | |
|---|---|
| NO. | Sequence |
| 1 | YₙXₙYₙXₙYₙ (where X is a positively charged |
| | amino acid such as arginine, histidine |
| | or lysine and Y is a spacer amino acid such |
| | as serine or glycine, and where each n |
| | is independently an integer 0 to 4) |
| 2 | YₙZₙYₙZₙYₙ (where Z is a negatively charged |
| | amino acid such as aspartic acid or |
| | glutamic acid and Y is a spacer amino acid |
| | such as serine or glycine, and where |
| | each n is independently an integer 0 to 4) |
| 3 | YYₙXXₙYYₙXXₙYYₙ (where X is a positively |
| | charged amino acid such as arginine, |
| | histidine or lysine and Y is a spacer |
| | amino acid such as serine or glycine, and |
| | where each n is independently an integer |
| | 0 to 4) |
| 4 | YYₙZZₙYYₙZZₙYYₙ (where Z is a negatively |
| | charged amino acid such as aspartic |
| | acid or glutamic acid and Y is a spacer |
| | amino acid such as serine or glycine, and |
| | where each n is independently an integer |
| | 0 to 4) |
| 5 | YₙXₙCYₙXₙYₙ (where X is a positively charged |
| | amino acid such as arginine, histidine |
| | or lysine and Y is a spacer amino acid such |
| | as serine or glycine, and where each n is |
| | independently an integer 0 to 4) |
| 6 | YₙZₙCYₙZₙYₙ (where Z is a negatively charged |
| | amino acid such as aspartic acid or |
| | glutamic acid and Y is a spacer amino acid |
| | such as serine or glycine, and where |
| | each n is independently an integer 0 to 4) |
| 7 | GSGSRKGGKRGS |
| 8 | GSGSRKCGKRGS |
| 9 | GSGSDEGGEDGS |
| 10 | GSGSDECGEDGS |

For example, in an embodiment, each of the first and second charge polarized core domains may comprise a peptide comprising the sequence YYₙXXₙYYₙXXₙYYₙ (where X is a positively charged amino acid such as arginine, histidine or lysine and Y is a spacer amino acid such as serine or glycine, and where each n is independently an integer 0 to 4; SEQ ID NO: 3). Illustrative peptide sequences include, but are not limited to, RKGGKR (SEQ ID NO: 11) or GSGSRKGGKRGS (SEQ ID NO: 12).

In another illustrative embodiment, each of the first and second charge polarized core domains may comprise a peptide comprising the sequence YYₙZZₙYYₙZZₙYYₙ (where Z is a negatively charged amino acid such as aspartic acid or glutamic acid and Y is a spacer amino acid such as serine or glycine, and where each n is independently an integer 0 to 4). Illustrative peptide sequences include, but are not limited to, DEGGED (SEQ ID NO: 13) or GSGSDEGGEDGS (SEQ ID NO: 14).

In one aspect, the present disclosure provides a heterodimeric protein comprising (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the heterodimeric protein comprises two individual polypeptide chains which self-associate. In embodiments, the linker facilitates heterodimerization. In embodiments, the heterodimeric protein comprises two of the same butyrophilin family proteins or two different butyrophilin family proteins. In embodiments, the butyrophilin family proteins comprise a V-type domain and/or a B30.2 domain. In embodiments, the first domain is a butyrophilin-like (BTNL) family protein, such as BTN1A1, BTN2A1, BTN2A2, BTN2A3, BTN3A1, BTN3A2, BTN3A3, BTNL2, BTNL3, BTNL8, BTNL9, BTNL10, and SKINTL.

Illustrative sequences of linkers that adjoins the first and second domain, also referred to herein as a core domain are provided below:

In embodiments, the core domain has the following sequence:

(SEQ ID NO: 15)
SKYGPPCPPCPAPEFLGGPSVFLFPPKPKDQLMISRTPEVTCVVVDVSQED
PEVQFNWYVDGVEVHNAKTKPREEQFNSTYRVVSVLTVLHQDWLSGKEYKC
KVSSKGLPSSIEKTISNATGQPREPQVYTLPPSQEEMTKNQVSLTCLVKGF
YPSDIAVEWESNGQPENNYKTTPPVLDSDGSFFLYSRLTVDKSRWQEGNVF
SCSVLHEALHNHYTQKSLSLSLGKIEGRMD.

In embodiments, the core domain has the following sequence:

(SEQ ID NO: 16)
CPPCPAPEFLGGPSVFLFPPKPKDQLMISRTPEVTCVVVDVSQEDPEVQFN
WYVDGVEVHNAKTKPREEQFNSTYRVVSVLTVLHQDWLSGKEYKCKVSSKG
LPSSIEKTISNATGQPREPQVYTLPPSQEEMTKNQVSLTCLVKGFYPSDIA
VEWESNGQPENNYKTTPPVLDSDGSFFLYSRLTVDKSRWQEGNVFSCSVLH
EALHNHYTQKSLSLSLGK.

In embodiments, the core domain is a KIHT22Y protein having the following sequence (T to Y mutations are indicated in an underlined, boldface font):

(SEQ ID NO: 17)
EPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLYCL
VKGFYPSDIAVEWESNGQPENNYKTTPPVLDSDGSFFLYSKLTVDKSRWQQ
GNVFSCSVMHEALHNHYTQKSLSLSPGK.

In embodiments, the core domain is a KIHY86T protein having the following sequence (Y to T mutations are indicated in an underlined, boldface font):

(SEQ ID NO: 18)
EPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCL
VKGFYPSDIAVEWESNGQPENNYKTTPPVLDSDGSFFLTSKLTVDKSRWQQ
GNVFSCSVMHEALHNHYTQKSLSLSPGK.

In embodiments, the core domain is a KIHY86T protein having the following sequence:

(SEQ ID NO: 19)
VPRDCGCKPCICTVPEVSSVFIFPPKPKDVLTITLTPKVTCVVVDISKDDP
EVQFSWFVDDVEVHTAQTQPREEQFNSTFRSVSELPIMHQDWLNGKEFKCR
VNSAAFPAPIEKTISKTKGRPKAPQVYTIPPPKEQMAKDKVSLTCMITDFF
PEDITVEWQWNGQPAENYKNTQPIMDTDGSYFVYSKLNVQKSNWEAGNTFT
CSVLHEGLHNHHTEKSLSHSPGI.

The sequence of an illustrative charge polarized core domain (positive-negative) is provided below (positively charged peptide is shown in an underlined font, negatively charged peptide is indicated in a boldface, underlined font):

(SEQ ID NO: 20)
GSGSRKGGKRGSKYGPP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

DEGGEDGSGS.

The sequence of an illustrative charge polarized core domain (negative-positive) is provided below (positively charged peptide is shown in an underlined font, negatively charged peptide is indicated in a boldface, underlined font):

(SEQ ID NO: 21)
GSGSDEGGEDGSKYGPP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

RKGGKRGSGS.

The sequence of an illustrative charge polarized core domain (negative-positive) is provided below:

(SEQ ID NO: 22)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

.

In various embodiments, the protein comprising the charged amino acid residues may further comprise one or more cysteine residues to facilitate disulfide bonding between the electrostatically charged core domains as an additional method to stabilize the heterodimer.

In various embodiments, each of the first and second charge polarized core domains comprises a linker sequence which may optionally function as a stabilizing domain. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker (e.g., a stabilizing domain) is a synthetic linker such as PEG.

In other embodiments, the linker (e.g., a stabilizing domain) is a polypeptide. In embodiments, the linker (e.g., a stabilizing domain) is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker (e.g., a stabilizing domain) may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the linker (e.g., a stabilizing domain) is substantially comprised of glycine and serine residues (e.g., about 30%, or about 40%, or about 50%, or about 60%, or about 70%, or about 80%, or about 90%, or about 95%, or about 97% glycines and serines).

In various embodiments, the linker (e.g., a stabilizing domain) is a hinge region of an antibody (e.g., of IgG, IgA, IgD, and IgE, inclusive of subclasses (e.g., IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In other embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys which, when dimerized by disulfide bond formation, results in a cyclic octapeptide believed to act as a pivot, thus conferring flexibility. In various embodiments, the present linker (e.g., a stabilizing domain) comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker (e.g., a stabilizing domain) of the present disclosure comprises one or more glycosylation sites.

In various embodiments, the linker (e.g., a stabilizing domain) comprises an Fc domain of an antibody (e.g., of IgG, IgA, IgD, and IgE, inclusive of subclasses (e.g., IgG1, IgG2, IgG3, and IgG4, and IgA1 and IgA2)). In various embodiments, the linker (e.g., a stabilizing domain) comprises a hinge-CH2-CH3 Fc domain derived from a human IgG4 antibody. In various embodiments, the linker (e.g., a stabilizing domain) comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present heterodimeric proteins.

In embodiments, the Fc domain contains one or more amino acid substitutions at amino acid residue 250, 252, 254, 256, 308, 309, 311, 428, 433 or 434 (in accordance with Kabat numbering), or equivalents thereof. In an embodiment, the amino acid substitution at amino acid residue 250 is a substitution with glutamine. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, orthreonine. In an embodiment, the amino acid substitution at amino acid residue 308 is a substitution with threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 428 is a substitution with leucine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In embodiments, the Fc domain (e.g., comprising an IgG constant region) comprises one or more mutations such as substitutions at amino acid residue 252, 254, 256, 433, 434, or 436 (in accordance with Kabat numbering). In an embodiment, the IgG constant region includes a triple M252Y/S254T/T256E mutation or YTE mutation. In another embodiment, the IgG constant region includes a triple H433K/N434F/Y436H mutation or KFH mutation. In a further embodiment, the IgG constant region includes an YTE and KFH mutation in combination.

In embodiments, the modified humanized antibodies of the invention comprise an IgG constant region that contains one or more mutations at amino acid residues 250, 253, 307, 310, 380, 428, 433, 434, and 435. Illustrative mutations include T250Q, M428L, T307A, E380A, I253A, H310A, M428L, H433K, N434A, N434F, N434S, and H435A. In an embodiment, the IgG constant region comprises a M428L/N434S mutation or LS mutation. In another embodiment, the IgG constant region comprises a T250Q/M428L mutation or QL mutation. In another embodiment, the IgG constant region comprises an N434A mutation. In another embodiment, the IgG constant region comprises a T307A/E380A/N434A mutation or AAA mutation. In another embodiment, the IgG constant region comprises an I253A/H310A/H435A mutation or IHH mutation.

In another embodiment, the IgG constant region comprises a H433K/N434F mutation. In another embodiment, the IgG constant region comprises a M252Y/S254T/T256E and a H433K/N434F mutation in combination.

In various embodiments, mutations are introduced to increase stability and/or half-life of the Fc domain. An illustrative Fc stabilizing mutant is S228P. Additional illustrative Fc half-life extending mutants are T250Q, M428L, V308T, L309P, and Q311S and the present linkers (e.g., stabilizing domains) may comprise 1, or 2, or 3, or 4, or 5 of these mutants.

Additional illustrative mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al., Nature (2014) 514:642-645, Grevys et al., Journal of Immunology. (2015), 194(11):5497-508, and U.S. Pat. No. 7,083,784, the entire contents of which are hereby incorporated by reference.

In various embodiments, the linker may be flexible, including without limitation highly flexible. In various embodiments, the linker may be rigid, including without limitation a rigid alpha helix.

In various embodiments, the linker may be functional. For example, without limitation, the linker may function to improve the folding and/or stability, improve the expression, improve the pharmacokinetics, and/or improve the bioactivity of the present heterodimeric protein. In another example, the linker may function to target the heterodimeric protein to a particular cell type or location.

The Heterodimeric Proteins

In one aspect, the present disclosure provides a heterodimeric protein, which is suitable for cancer therapy of the present disclosure, the heterodimeric protein comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain.

In embodiments the heterodimeric protein is a complex of two polypeptide chains.

In one aspect, the present disclosure provides a heterodimeric protein, which is suitable for cancer therapy of the present disclosure, the heterodimeric protein comprises an alpha chain and a beta chain wherein the alpha chain and the beta chain each independently comprise (a) a first domain comprising a butyrophilin family protein, or fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain.

In embodiments the alpha chain and the beta chain self-associate to form the heterodimer.

In embodiments, the first domain comprises two of the same butyrophilin family proteins. In embodiments, wherein the first domain comprises two different butyrophilin family proteins. In embodiments, the butyrophilin family proteins comprise a V-type domain. In embodiments, the butyrophilin family proteins or fragments thereof are derived from the native butyrophilin family proteins that comprise a B30.2 domain in the cytosolic tail.

In embodiments, the butyrophilin family proteins are selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the first domain comprises: (a) any one of BTN1A1, BTN2A1, BTN2A2, BTN2A3, BTN3A1, BTN3A2, BTN3A3, BTNL2, BTNL3, BTNL8, BTNL9, BTNL10, and SKINTL; and (b) any one of BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the first domain comprises: (a) any one of human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL, and (b) any one of human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In embodiments, the first domain comprises a fragment of butyrophilin family proteins, wherein the fragment is capable of binding a gamma delta T cell receptor and is optionally an extracellular domain, optionally comprising one or more of an immunoglobulin V (IgV)- and IgC-like domain. In embodiments, the first domain comprises a fragment of butyrophilin family proteins, wherein the fragment is capable of binding a gamma delta T cell receptor selected from a Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In embodiments, the first domain comprises a polypeptide having an amino acid sequence of: (a) a polypeptide having an amino acid sequence selected from the amino acid sequence of SEQ ID NOs: 24 to 45, or a fragment thereof; and (b) a polypeptide having an amino acid sequence selected from the amino acid sequence of SEQ ID NOs: 24 to 45, or a fragment thereof. In embodiments, the linker comprises a polypeptide having an amino acid sequence selected from the amino acid sequence of SEQ ID NOs: 1-14. In embodiments, the linker comprises a polypeptide having an amino acid sequence selected from the amino acid sequence of SEQ ID NOs: 15-22.

Additionally, or alternatively, in embodiments, in the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2. In embodiments, the antibody-like molecule is an scFv. In embodiments, the targeting domain is an extracellular domain. In embodiments, the targeting domain is capable of binding an antigen on the surface of a cancer cell. In embodiments, the targeting domain specifically binds a protein selected from CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, CDH18, EPHA3, NY-eso-2, MAGE-A10, MAGE-A3, MAGE-A7, HER3, and GD3. In embodiments, the targeting domain comprises a portion of the extracellular domain of LAG-3, PD-1, TIGIT, CD19, or PSMA. In embodiments, the targeting domain specifically binds CD19. In embodiments, the targeting domain specifically binds PSMA. In embodiments, the targeting domain specifically binds CD33. In embodiments, the targeting domain specifically binds CD20. In embodiments, the targeting domain specifically binds CLL-1.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linkerforms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally human IgG1. In embodiments, the linker comprises the hinge-CH2-CH3 Fc domain derived from IgG4, optionally human IgG4. In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains.

In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12). In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YnZnYnZnYn (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6). In embodiments, the peptide comprising negatively charged amino acid residues comprises the sequence DEGGED (SEQ ID NO: 13) or GSGSDEGGEDGS (SEQ ID NO: 14).

Additionally or alternatively, in embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ754. In embodiments, the first domain comprises BTNL3 and BTNL8 and it modulates a Vγ4-expressing T cell. In embodiments, the first domain comprises BTNL2A1 and BTNL3A1 and it modulates a Vγ9δ2-expressing T cell. In embodiments, the first domain comprises BTNL3A1 and BTNL3A2 and it modulates a Vγ9δ2-expressing T cell. In embodiments, the first domain comprises BTNL3A1 and BTNLA3 and it modulates a Vγ9δ2-expressing T cell. In embodiments, the first domain comprises BTNL1 and BTNL6 and it modulates a Vγ754-expressing T cell. In embodiments, the modulation of a gamma delta T cell is activation of a gamma delta T cell.

Additionally or alternatively, in embodiments, the heterodimeric protein is capable of forming a synapse between a gamma delta T cell and a tumor cell. In embodiments, the heterodimeric protein is capable of contemporaneous activation and targeting of gamma delta T cells to tumor cells.

In embodiments, the present heterodimeric proteins comprise a portion of a butyrophilin-like (BTNL) proteins. In an illustrative embodiment, the first domain is a butyrophilin-like (BTNL) family protein. Examples of BTNL family proteins include BTNL1, BTNL3, BTNL8, BTN3A1, BTN3A2, and BTN3A3. In embodiments, the heterotrimeric protein modulates the function of gamma delta T cells. In embodiments, in addition to the BTNL family protein, the heterodimeric proteins further comprise a portion of the extracellular domain of LAG-3, PD-1, or TIGIT and which is capable of binding its receptor/ligand on the surface of a cancer cell. In embodiments, in addition to the BTNL family protein, the heterodimeric proteins further comprise an antibody or fragment thereof (e.g., comprising a portion of the antigen-binding domain of an antibody and/or a CDR3 that binds a tumor epitope) and which is capable of binding an antigen on the surface of a cancer cell.

In embodiments, the present heterodimeric proteins comprise a portion of a butyrophilin-like (BTNL) proteins. In an illustrative embodiment, the first domain is a butyrophilin-like (BTNL) family protein. Examples of BTNL family proteins include BTNL1, BTNL3, BTNL8, BTN3A1, BTN3A2, and BTN3A3. In embodiments, the 5 heterotrimeric protein modulates the function of gamma delta T cells. In embodiments, in addition to the BTNL family protein, the heterodimeric proteins further comprise a portion of the extracellular domain of LAG-3, PD-1, TIGIT, CD19, PSMA, or antibody-derived binding domain (e.g. CDR3, Fab, scFv domain, etc.) targeting a tumor antigen (such as CD19 or PSMA) and which is capable of binding its receptor/ligand on the surface of a cancer cell. In embodiments, in addition to the BTNL family protein, the heterodimeric proteins further comprise an antibody or fragment thereof (e.g., comprising a portion of the antigen-binding domain of an antibody) and which is capable of binding an antigen on the surface of a cancer cell.

In embodiments, the second domain is a LAG-3 protein.

In embodiments, the second domain is a PD-1 protein.

In embodiments, the second domain is a TIGIT protein.

In embodiments, the second domain is a CD19 protein binding domain, such as an scFv, CDR3, or Fab. In embodiments, the second domain is a CD19 protein and the heterodimeric protein further comprise an antibody or fragment thereof (e.g., comprising a portion of the antigen-binding domain of an antibody) and which is capable of binding an antigen on the surface of a cancer cell.

In embodiments, the second domain is a PSMA protein binding domain, such as an scFv, CDR3, or Fab. In embodiments, the second domain is a PSMA protein and the heterodimeric protein further comprise an antibody or fragment thereof (e.g., comprising a portion of the antigen-binding domain of an antibody) and which is capable of binding an antigen on the surface of a cancer cell.

In an illustrative embodiment, the second domain is a receptorfor EGP such as EGFR (ErbB1), ErbB2, ErbB3 and ErbB4.

In an illustrative embodiment, the second domain is a receptor for insulin or an insulin analog such as the insulin receptor and/or IGF1 or IGF2 receptor.

In an illustrative embodiment, the second domain is a receptor for EPO such as the EPO receptor (EPOR) receptor and/or the ephrin receptor (EphR).

In various embodiments, the heterodimeric protein may comprise a domain of a soluble (e.g., non-membrane associated) protein. In various embodiments, the heterodimeric protein may comprise a fragment of the soluble protein which is involved in signaling (e.g., a portion of the soluble protein which interacts with a receptor).

In various embodiments, the heterodimeric protein may comprise the extracellular domain of a transmembrane protein. In various embodiments, one of the extracellular domains transduces an immune inhibitory signal and one of the extracellular domains transduces an immune stimulatory signal.

In embodiments, an extracellular domain refers to a portion of a transmembrane protein which is capable of interacting with the extracellular environment. In various embodiments, an extracellular domain refers to a portion of a transmembrane protein which is sufficient to bind to a ligand or receptor and effective transmit a signal to a cell. In various embodiments, an extracellular domain is the entire amino acid sequence of a transmembrane protein which is external of a cell or the cell membrane. In various embodiments, an extracellular domain is the that portion of an amino acid sequence of a transmembrane protein which is external of a cell or the cell membrane and is needed for signal transduction and/or ligand binding as may be assayed using methods know in the art (e.g., in vitro ligand binding and/or cellular activation assays).

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes. Specific examples include direct stimulation of cytokine receptors such as IL-2R, IL-7R, IL-15R, IL-17R or IL-21R using fusion proteins encoding the ligands for such receptors (IL-2, IL-7, IL-15, IL-17 or IL-21, respectively). Stimulation from any one of these receptors may directly stimulate the proliferation and cytokine production of individual T cell subsets.

In embodiments, the extracellular domain or antibody binding domain (e.g. CDR3, Fab, scFv domain, etc.) may be used to produce a soluble protein to competitively inhibit signaling by that receptor's ligand. For instance, without limitation, competitive inhibition of PD-L1 or PD-L2 could be achieved using PD-1, or competitive inhibition of PVR could be achieved using TIGIT. In embodiments, the extracellular domain or antibody binding domain (e.g. CDR3, Fab, scFv domain, etc.) may be used to provide artificial signaling.

In embodiments, the present heterodimeric proteins deliver or mask an immune inhibitory signal. In embodiments, the present heterodimeric proteins deliver or mask an immune stimulatory signal.

In various embodiments, the present heterodimeric proteins may be engineered to target one or more molecules that reside on human leukocytes including, without limitation, the extracellular domains (where applicable) of SLAMF4, IL-2Rα, IL-2 R β, ALCAM, B7-1, IL-4 R, B7-H3, BLAME/SLAMFS, CEACAM1, IL-6 R, IL-7 Rα, IL-10R α, IL-I 0 R β, IL-12 R β1, IL-12 R β2, CD2, IL-13 R α 1, IL-13, CD3, CD4, ILT2/CDS5j, ILT3/CDS5k, ILT4/CDS5d, ILT5/CDS5α, lutegrin a 4/CD49d, CD6, Integrin α E/CD103, CD6, Integrin α M/CD 11 b, CDS, Integrin α X/CD11c, Integrin β 2/CDIS, KIR/CD15S, KIR2DL1, CD2S, KIR2DL3, KIR2DL4/CD15Sd, CD31/PECAM-1, KIR2DS4, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, CX3CR1, CX3CL1, L-Selectin, SIRPβ1, SLAM, TCCR/WSX-1, DNAM-1, Thymopoietin, EMMPRIN/CD147, TIM-1, EphB6, TIM-2, TIM-3, TIM-4, Fcγ RIII/CD16, TIM-6, Granulysin, ICAM-1/CD54, ICAM-2/CD102, IFN-γR1, IFN-γ R2, TSLP, IL-1 R1 and TSLP R.

In embodiments, the present heterodimeric proteins may be engineered to target one or more molecules involved in immune inhibition, including for example: CTLA-4, PD-L1, PD-L2, PD-1, BTLA, HVEM, TIM3, GAL9, LAG3, VISTANSIG8, KIR, 2B4, TIGIT, CD160 (also referred to as BY55), CHK 1 and CHK2 kinases, A2aR, CEACAM (e.g., CEACAM-1, CEACAM-3 and/or CEACAM-5), and various B-7 family ligands (including, but are not limited to, B7-1, B7-2, B7-DC, B7-H1, B7-H2, B7-H3, B7-H4, B7-H5, B7-H6 and B7-H7).

In embodiments, the present heterodimeric proteins comprise an extracellular domain of an immune inhibitory agent. In embodiments, the present heterodimeric proteins comprise an antibody binding domain (e.g. CDR3, Fab, scFv domain, etc.) directed against an immune inhibitory agent.

In embodiments, the present heterodimeric proteins comprise an extracellular domain of a soluble or membrane protein which has immune inhibitory properties. In embodiments, the present heterodimeric proteins comprise an antibody binding domain (e.g. CDR3, Fab, scFv domain, etc.) which has immune inhibitory properties In embodiments, the present heterodimeric proteins simulate binding of an inhibitory signal ligand to its cognate receptor but inhibit the inhibitory signal transmission to an immune cell (e.g., a T cell, macrophage or other leukocyte).

In various embodiments, the heterodimeric protein comprises an immune inhibitory receptor extracellular domain or antibody binding domain (e.g. CDR3, Fab, scFv domain, etc.) and an immune stimulatory ligand extracellular domain or antibody binding domain (e.g. CDR3, Fab, scFv domain, etc.) which can, without limitation, deliver an immune stimulation to a T cell while masking a tumor cell's immune inhibitory signals. In various embodiments, the heterodimeric protein delivers a signal that has the net result of T cell activation.

In embodiments, the present heterodimeric proteins comprise an extracellular domain of a soluble or membrane protein which has immune stimulatory properties. In embodiments, the present heterodimeric proteins comprise an antibody binding domain (e.g. CDR3, Fab, scFv domain, etc.) which has immune stimulatory properties.

In various embodiments, the present heterodimeric protein may comprise variants of any of the known cytokines, growth factors, and/or hormones. In various embodiments, the present heterodimeric proteins may comprise variants of any of the known receptors for cytokines, growth factors, and/or hormones. In various embodiments, the present heterodimeric proteins may comprises variants of any of the known extracellular domains, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the known amino acid or nucleic acid sequences.

In various embodiments, the present heterodimeric protein may comprise an amino acid sequence having one or more amino acid mutations relative to any of the known protein sequences. In embodiments, the one or more amino acid mutations may be independently selected from substitutions, insertions, deletions, and truncations.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, IIe; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the heterodimeric proteins by reference to the genetic code, including taking into account codon degeneracy.

In any of these sequence, the core domain having the amino acid sequence that is or comprises an amino acid sequence having at least 90%, or at least 91%, or at least 92%, or at least 93%, or at least 94%, or at least 95%, or at least 96%, or at least 97%, or at least 98%, or at least 98%, or at least 99% (e.g. about 90%, or about 91%, or about 92%, or about 93%, or about 94%, or about 95%, or about 96%, or about 97%, or about 98%, or about 98%, or about 99%) sequence identity to an amino acid sequence selected from the amino acid sequence of SEQ ID NOs: 15-22.

In various embodiments, the present heterodimeric proteins are capable of, and can be used in methods comprising, promoting immune activation (e.g., against tumors). In various embodiments, the present heterodimeric proteins are capable of, and can be used in methods comprising, suppressing immune inhibition (e.g., that allows tumors to survive). In various embodiments, the present heterodimeric protein provides improved immune activation and/or improved suppression of immune inhibition.

In various embodiments, the present heterodimeric proteins are capable of, or can be used in methods comprising, modulating the amplitude of an immune response, e.g., modulating the level of effector output.

In embodiments, e.g., when used for the treatment of cancer, the present heterodimeric protein alters the extent of immune stimulation as compared to immune inhibition to increase the amplitude of a T cell response, including, without limitation, stimulating increased levels of cytokine production, proliferation or target killing potential.

In embodiments, a subject is further administered autologous or allogeneic gamma delta T cells that were expanded ex vivo.

In embodiments, a subject is further administered autologous or allogeneic T cells that express a Chimeric Antigen Receptor (i.e., CAR-T cells). CAR-T cells are described in, as examples, Eshhar, et al., PNAS USA. 90(2):720-724, 1993; Geiger, et al., J Immunol. 162(10):5931-5939, 1999; Brentjens, et al., Nat Med. 9(3):279-286, 2003; Cooper, et al., Blood 101(4):1637-1644, 2003; Imai, et al., Leukemia. 18:676-684, 2004, Pang, et al., Mol Cancer. 2018; 17:91, and Schmidts, et al., Front. Immunol 2018; 9:2593; the entire contents of which are hereby incorporated by reference.

In embodiments, the heterodimeric proteins act synergistically when used in combination with Chimeric Antigen Receptor (CAR) T-cell therapy. In an illustrative embodiment, the heterodimeric proteins act synergistically when used in combination with CAR T-cell therapy in treating a tumor or cancer. In an embodiment, the heterodimeric proteins act synergistically when used in combination with CAR T-cell therapy in treating blood-based tumors. In an embodiment, the heterodimeric proteins act synergistically when used in combination with CAR T-cell therapy in treating solid tumors. For example, use of heterodimeric proteins and CAR T-cells may act synergistically to reduce or eliminate the tumor or cancer, or slow the growth and/or progression and/or metastasis of the tumor or cancer. In various embodiments, the heterodimeric proteins of the invention induce CAR T-cell division. In various embodiments, the heterodimeric proteins of the invention induce CAR T-cell proliferation. In various embodiments, the heterodimeric proteins of the invention prevents anergy of the CAR T cells.

In various embodiments, the CAR T-cell therapy comprises CAR T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MET, MUC1, MUC16, ROR-1, IL13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, GPC3, human epidermal growth factor receptor 2 (HER2), K light chain, mesothelin, EGFR, EGFRvIII, ErbB, fibroblast activation protein (FAP), carcinoembryonic antigen (CEA), PMSA, Receptor Tyrosine Kinase Like Orphan Receptor 1 (ROR1), TAG72, and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, am11, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Exemplary CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In embodiments, the CAR-T cells are autologous or allogeneic gamma delta T cells.

In various embodiments the present heterodimeric proteins, in embodiments are capable of, or find use in methods involving, masking an inhibitory ligand on the surface of a tumor cell and replacing that immune inhibitory ligand with an immune stimulatory ligand. Accordingly, the present heterodimeric proteins, in embodiments are capable of, or find use in methods involving, reducing or eliminating an inhibitory immune signal and/or increasing or activating an immune stimulatory signal. For example, a tumor cell bearing an inhibitory signal (and thus evading an immune response) may be substituted for a positive signal binding on a T cell that can then attack a tumor cell. Accordingly, in embodiments, an inhibitory immune signal is masked by the present heterodimeric proteins and a stimulatory immune signal is activated. Such beneficial properties are enhanced by the single construct approach of the present heterodimeric proteins. For instance, the signal replacement can be effected nearly simultaneously and the signal replacement is tailored to be local at a site of clinical importance (e.g., the tumor microenvironment).

In various embodiments, the present heterodimeric proteins are capable of, or find use in methods comprising, stimulating or enhancing the binding of immune stimulatory receptor/ligand pairs.

In other embodiments, the present heterodimeric proteins are capable of, or find use in methods involving, enhancing, restoring, promoting and/or stimulating immune modulation. In embodiments, the present heterodimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the present heterodimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: proinflammatory cytokine production or T cell migration or T cell tumor infiltration.

In embodiments, the present heterodimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the present heterodimeric proteins are capable of, or find use in methods involving, inhibiting and/or causing a decrease in recruitment of immunosuppressive cells (e.g., myeloid-derived suppressor cells (MDSCs), regulatory T cells (Tregs), tumor associated neutrophils (TANs), M2 macrophages, and tumor associated macrophages (TAMs)) to the tumor and/or tumor microenvironment (TME). In embodiments, the present therapies may alter the ratio of M1 versus M2 macrophages in the tumor site and/or TME to favor M1 macrophages.

In embodiments, the heterotrimeric protein modulates the function of gamma delta T cells.

In various embodiments, the present heterodimeric proteins are capable of, and can be used in methods comprising, inhibiting and/or reducing T cell inactivation and/or immune tolerance to a tumor, comprising administering an effective amount of a heterodimeric protein described herein to a subject. In embodiments, the present heterodimeric proteins are able to increase the serum levels of various cytokines including, but not limited to, one or more of IFNγ, IL-2, IL-4, IL-5, IL-6, IL-9, IL-10, IL-13, IL-17A, IL-17F, and IL-22. In embodiments, the present heterodimeric proteins are capable of enhancing IL-2, IL-4, IL-5, IL-10, IL-13, IL-17A, IL-22, or IFNγ in the serum of a treated subject.

In various embodiments, the present heterodimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during cancer. This dysfunction is defined by poor proliferative and/or effector functions, sustained expression of inhibitory receptors and a transcriptional state distinctfrom that of functional effector or memory T cells. Exhaustion prevents optimal control of tumors. In addition, an anti-tumor CD8+ and/or CD4+ T cell refers to T cells that can mount an immune response to a tumor. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refers to receptors (e.g., CTLA-4, B7-H3, B7-H4, TIM-3) expressed on immune cells that prevent or inhibit uncontrolled immune responses.

In various embodiments, the present heterodimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL-7R/CD127⁺); CD8⁺ effector T cells (e.g., αβ TCR, CD3⁺, CD8⁺, CCR7⁺, CD62Lhi, IL-7R/CD127⁺); effector memory T cells (e.g., CD62Llow, CD44⁺, TCR, CD3⁺, IL-7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻ CD62L⁻) (TemE and TemL, respectively); CD127(⁺)CD25(low/−) effector T cells; CD127(−)CD25(−) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(−) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25-regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells),T helper type 3 (Th3) cells, regulatory cell of natural killer T cell phenotype (NKTregs), CD8⁺ regulatory T cells, CD8⁺CD28− regulatory T cells and/or regulatory T-cells secreting IL-10, IL-35, TGF-β, TNF-α, Galectin-1, IFN-γ and/or MCP1.

In various embodiments, the present heterodimeric proteins are capable of, and can be used in methods comprising, transiently stimulating effector T cells for no longer than about 12 hours, about 24 hours, about 48 hours, about 72 hours or about 96 hours or about 1 week or about 2 weeks. In various embodiments, the present heterodimeric proteins are capable of, and can be used in methods comprising, transiently depleting or inhibiting regulatory T cells for no longer than about 12 hours, about 24 hours, about 48 hours, about 72 hours or about 96 hours or about 1 week or about 2 weeks. In various embodiments, the transient stimulation of effector T cells and/or transient depletion or inhibition of regulatory T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In various embodiments, the present heterodimeric proteins provide advantages including, without limitation, ease of use and ease of production. This is because two distinct immunotherapy agents are combined into a single product which allows for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present heterodimeric proteins are easier and more cost effective to manufacture.

In various embodiments, the present heterodimeric proteins provide synergistic therapeutic effects as it allows for improved site-specific interplay of two immunotherapy agents. In embodiments, the present heterodimeric proteins provide the potential for reducing off-site and/or systemic toxicity.

Diseases

In one aspect, the present disclosure provides a method of treating cancer, comprising administering to a subject in need thereof an effective amount of a pharmaceutical composition of any of the embodiments disclosed herein to a subject in need thereof. In embodiments, the cancer is a lymphoma. In embodiments, the cancer is a leukemia. In embodiments, the cancer is a Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In embodiments, the cancer is prostate cancer. In embodiments, the cancer is an epithelial-derived carcinoma. In embodiments, the cancer is known to express the antigenic target of the second domain of the heterodimeric protein. In embodiments, the cancer is known to contain mutations which limit recognition by alpha beta T cells, including but not limited to mutations in MHC I, beta 2 microglobulin, TAP, etc.

In embodiments, the subject is further administered autologous or allogeneic gamma delta T cells that were expanded ex vivo. In embodiments, the autologous or allogeneic gamma delta T cells express a Chimeric Antigen Receptor. In embodiments, the subject is further administered autologous or allogeneic T cells that express a Chimeric Antigen Receptor.

In various embodiments, the present disclosure pertains to cancers and/or tumors; for example, the treatment or prevention of cancers and/or tumors. As described elsewhere herein, the treatment of cancer may involve in various embodiments, modulating the immune system with the present heterodimeric proteins to favor immune stimulation over immune inhibition.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system. The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in alocal area, forming a new tumor, which may be alocal metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present disclosure include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the cancer is an epithelial-derived carcinoma.

In embodiments, the heterodimeric protein is used to treat a subject that has a treatment-refractory cancer. In embodiments, the heterodimeric protein is used to treat a subject that is refractory to one or more immune-modulating agents. For example, in embodiments, the heterodimeric protein is used to treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g., ipilimumab (YERVOY)-refractory patients (e.g., melanoma patients). Accordingly, in various embodiments the present disclosure provides methods of cancer treatment that rescue patients that are non-responsive to various therapies, including monotherapy of one or more immune-modulating agents.

In various embodiments, the present disclosure provides heterodimeric proteins which target a cell or tissue within the tumor microenvironment. In embodiments, the cell or tissue within the tumor microenvironment expresses one or more targets or binding partners of the heterodimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In various embodiments, the present heterodimeric protein targets a cancer cell. In embodiments, the cancer cell expresses one or more of targets or binding partners of the heterodimeric protein.

In various embodiments, the heterodimeric protein of the invention may target a cell (e.g., cancer cell or immune cell) that expresses any of the receptors as described herein. For example, the heterodimeric protein of the invention may target a cell that expresses any of the receptors for a cytokine, growth factor, and/or hormone as described herein.

In embodiments, the present methods provide treatment with the heterodimeric protein in a patient who is refractory to an additional agent, such “additional agents” being described elsewhere herein, inclusive, without limitation, of the various chemotherapeutic agents described herein.

In some aspects, the present chimeric agents are used in methods of activating a T cell, e.g., via the extracellular domain having an immune stimulatory signal or antibody binding domain (e.g. CDR3, Fab, scFv domain, etc.) having an immune stimulatory signal.

Methods of Treatment, and Patient Selections

In one aspect, the present disclosure relates to a method for identifying a cancer therapy, the method comprising: (a) obtaining a biological sample, the sample comprising one or more gamma/delta T cells; (b) evaluating the sample for the presence, absence, or level of one or more gamma/delta T-cell receptor (TCR) chains; and (c) selecting a cancer therapy having an ability to signal at the gamma/delta TCR, the cancer therapy comprising one or more butyrophilin proteins, or a fragment thereof. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In one aspect, the present disclosure relates to a method for selecting a patient for a cancer treatment, the method comprising: (a) obtaining a biological sample, the sample comprising one or more gamma/delta T cells; (b) evaluating the sample for the presence, absence, or level of one or more gamma/delta T-cell receptor (TCR) chains; and (C) selecting a cancer therapy having an ability to signal at the gamma/delta TCR, the cancer therapy comprising one or more butyrophilin proteins, or a fragment thereof. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ, Vδ6, Vδ7, and Vδ8.

In one aspect, the present disclosure relates to a method for making an agent for the treatment of a cancer in a cancer patient, comprising: (a) obtaining the agent for the treatment of a cancer, the obtaining comprising: (i) evaluating the biological sample from a cancer patient for the presence, absence, or level of one or more gamma/delta T-cell receptor (TCR) chains; and (ii) selecting a cancer therapy having an ability to signal at the one or more gamma/delta T-cell receptor (TCR) chains, the cancer therapy comprising one or more butyrophilin proteins, or a fragment thereof; (b) formulating the identified agent for administration to a cancer patient. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In one aspect, the present disclosure relates to a method for identifying a cancer therapy for a tumor, the method comprising: (a) obtaining a biological sample, the sample comprising one or more gamma/delta T cells; (b) evaluating the sample for the presence, absence, or level of one or more butyrophilin proteins; and (c) selecting a cancer therapy having an ability to signal at a gamma/delta TCR, the cancer therapy comprising the one or more butyrophilin proteins, or a fragment thereof. In embodiments, the evaluating comprises measuring the presence, absence, or level of one or more butyrophilin proteins selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the evaluating comprises measuring the presence, absence, or level of one or more butyrophilin proteins selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In one aspect, the present disclosure relates to a method for selecting a patient for a cancer treatment, the method comprising: (a) obtaining a biological sample, the sample comprising one or more gamma/delta T cells; (b) evaluating the sample for the presence, absence, or level of one or more butyrophilin proteins; and (c) selecting a cancer therapy having an ability to signal at a gamma/delta TCR, the cancer therapy comprising the one or more butyrophilin proteins, or a fragment thereof. In embodiments, the evaluating comprises measuring the presence, absence, or level of one or more butyrophilin proteins selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the evaluating comprises measuring the presence, absence, or level of one or more butyrophilin proteins selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In one aspect, the present disclosure relates to a method for making an agent for the treatment of a cancer in a cancer patient, comprising: (a) obtaining the agent for the treatment of a cancer, the obtaining comprising: (i) evaluating the biological sample from a cancer patient for the presence, absence, or level of one or more butyrophilin proteins; and (ii) selecting a cancer therapy having an ability to signal at a gamma/delta TCR, the cancer therapy comprising the one or more butyrophilin proteins, or a fragment thereof; (b) formulating the identified agent for administration to a cancer patient. In embodiments, the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

In any of the embodiments disclosed herein, the biological sample is a tissue sample. In embodiments, the tissue sample is selected from fresh tissue sample, frozen tumor tissue specimen, cultured cells, circulating tumor cells, and a formalin-fixed paraffin-embedded tumor tissue specimen. In embodiments, the tissue sample is a biopsy sample. In embodiments, the biopsy sample is selected from endoscopic biopsy, bone marrow biopsy, endoscopic biopsy (e.g. cystoscopy, bronchoscopy and colonoscopy), needle biopsy (e.g. fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g. shave biopsy, punch biopsy, and incisional biopsy) and surgical biopsy. In embodiments, the tissue sample is selected from bone, bone marrow, lung, brain, liver, adrenal gland, colon, intestine, esophagus, pancreas, urinary bladder, breast, lymph node, and skin.

In any of the embodiments disclosed herein, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In any of the embodiments disclosed herein, the biological sample is a tumor sample. In embodiments, the tumor is metastatic. In embodiments, the tumor has metastasized to a tissue or an organ. In embodiments, the tissue or the organ is selected from bone, bone marrow, lung, brain, liver, adrenal gland, colon, intestine, esophagus, pancreas, urinary bladder, breast, lymph node, and skin. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In some embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), Meigs' syndrome cancer; renal carcinoma; colorectal cancer; and adrenal cancer.

In any of the embodiments disclosed herein, the evaluating is performed by DNA sequencing, RNA sequencing, immunohistochemical staining, western blotting, in cell western, immunofluorescent staining, ELISA, and fluorescent activating cell sorting (FACS) or a combination thereof.

In embodiments, the evaluating is performed by contacting the sample with an agent that specifically binds to one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8. In embodiments, the agent that specifically binds to one or more TCR is an antibody or fragment thereof. In embodiments, the antibody is a recombinant antibody, a monoclonal antibody, a polyclonal antibody, or fragment thereof. In embodiments, the evaluating is performed by contacting the sample with an antibody or fragment thereof that specifically binds to the one or more gamma/delta T-cell receptor (TCR) chains, and detecting the one or more gamma/delta T-cell receptor (TCR) chains using immunohistochemical staining, western blotting, in cell western, immunofluorescent staining, ELISA, and fluorescent activating cell sorting (FACS) or a combination thereof.

In any of the embodiments disclosed herein, the evaluating is performed by contacting the sample with an agent that specifically binds to one or more butyrophilin selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the evaluating is performed by contacting the sample with an agent that specifically binds to one or more butyrophilin selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. In embodiments, the agent that specifically binds to one or more butyrophilin is an antibody or fragment thereof. In embodiments, the antibody is a recombinant antibody, a monoclonal antibody, a polyclonal antibody, or fragment thereof. In embodiments, the evaluating is performed by contacting the sample with an antibody or fragment thereof that specifically binds to the one or more butyrophilin, and detecting the one or more butyrophilin using immunohistochemical staining, western blotting, in cell western, immunofluorescent staining, ELISA, and fluorescent activating cell sorting (FACS) or a combination thereof.

In any of the embodiments disclosed herein, the evaluating is performed by contacting the sample with an agent that specifically binds to one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8. In embodiments, the agent that specifically binds to one or more TCR is an antibody or fragment thereof. In embodiments, the antibody is a recombinant antibody, a monoclonal antibody, a polyclonal antibody, or fragment thereof.

In any of the embodiments disclosed herein, the level and/or activity of one or more butyrophilin is measured by contacting the sample with an agent that specifically binds to one or more of the nucleic acids. In embodiments, the agent that specifically binds to one or more of the nucleic acids is a nucleic acid primer or probe.

In any of the embodiments disclosed herein, the level and/or activity of one or more TCR is measured by contacting the sample with an agent that specifically binds to one or more of the nucleic acids. In embodiments, the agent that specifically binds to one or more of the nucleic acids is a nucleic acid primer or probe.

In embodiments, when the presence, or higher level of: TCR Vγ9δ2 is detected in tumor compared to healthy tissue or sample from an healthy individual, the cancer therapy comprising human BTN2A1, BTN3A1, BTN2A2 and/or BTN2A3 butyrophilin protein, or a fragment thereof is selected; and/or TCR Vδ4 is detected in tumor compared to healthy tissue or sample from an healthy individual, the cancer therapy comprising human BTNL3 butyrophilin protein, or a fragment thereof is selected.

In embodiments, when presence, or higher level of: human BTN1A1 is detected in tumor compared to healthy tissue or sample from an healthy individual, the cancer therapy comprising human BTN1A1 butyrophilin protein, or a fragment thereof is selected; human BTNL2 is detected in tumor compared to healthy tissue or sample from an healthy individual, the cancer therapy comprising human BTNL2 butyrophilin protein, or a fragment thereof is selected; human BTN2A1 is detected in tumor compared to healthy tissue or sample from an healthy individual, the cancer therapy comprising human BTN2A1 butyrophilin protein, or a fragment thereof is selected; human BTN2A2 is detected in tumor compared to healthy tissue or sample from an healthy individual, the cancer therapy comprising human BTN2A2 butyrophilin protein, or a fragment thereof is selected; human BTN2A3 is detected in tumor compared to healthy tissue or sample from an healthy individual, the cancer therapy comprising human BTN2A3 butyrophilin protein, or a fragment thereof is selected; human BTN2A3 is detected in tumor compared to healthy tissue or sample from an healthy individual, the cancer therapy comprising human BTN2A3 butyrophilin protein, or a fragment thereof is selected; human BTN3A1 is detected in tumor compared to healthy tissue or sample from an healthy individual, the cancer therapy comprising human BTN3A1 butyrophilin protein, or a fragment thereof is selected; human BTN3A2 is detected in tumor compared to healthy tissue or sample from an healthy individual, the cancer therapy comprising human BTN3A2 butyrophilin protein, or a fragment thereof is selected; human BTN3A3 is detected in tumor compared to healthy tissue or sample from an healthy individual, the cancer therapy comprising human BTN3A3 butyrophilin protein, or a fragment thereof is selected; human BTNL8 is detected in tumor compared to healthy tissue or sample from an healthy individual, the cancer therapy comprising human BTNL8 butyrophilin protein, or a fragment thereof is selected; human BTNL9 is detected in tumor compared to healthy tissue or sample from an healthy individual, the cancer therapy comprising human BTNL9 butyrophilin protein, or a fragment thereof is selected; human BTNL10 is detected in tumor compared to healthy tissue or sample from an healthy individual, the cancer therapy comprising human BTNL10 butyrophilin protein, or a fragment thereof is selected; and/or human SKINTL is detected in tumor compared to healthy tissue or sample from an healthy individual, the cancer therapy comprising human SKINTL butyrophilin protein, or a fragment thereof is selected.

In embodiments, when presence, or higher level of a butyrophilin family protein is observed in a tissue compared to another tissue, a cancer therapy comprising the butyrophilin family protein, or a fragment thereof is selected for treating a tumor metastasized in the tissue.

In embodiments, when presence, or higher level of a butyrophilin family protein is observed in a type of cancer (e.g. breast cancer, colorectal cancer, pancreatic cancer, prostate cancer, etc.) compared to normal tissue or another type of cancer, a cancer therapy comprising the butyrophilin family protein, or a fragment thereof is selected for treating the type of cancer.

In embodiments, when presence, or higher level of a specific γδ TCR is observed in a tissue compared to another tissue, a cancer therapy comprising one or more butyrophilin family protein, or a fragment thereof that binds to the specific γδ TCR is selected for treating a tumor metastasized in the tissue.

In embodiments, when presence, or higher level of a specific γδ TCR is observed in a type of cancer (e.g. breast cancer, colorectal cancer, pancreatic cancer, prostate cancer, etc.) compared to normal tissue or another type of cancer, a cancer therapy comprising one or more butyrophilin family protein, or a fragment thereof that binds to the specific γδ TCR for treating the type of cancer.

In any of the embodiments disclosed herein, the cancer therapy comprises a heterodimeric protein comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the first domain comprises two of the same butyrophilin family proteins. In embodiments, the first domain comprises two different butyrophilin family proteins. In embodiments, the butyrophilin family proteins, or a fragment thereof comprise an Ig-like V-type domain. In embodiments, the butyrophilin family proteins, or a fragment thereof are derived from native full length proteins. In embodiments, the first domain comprises one or more fragments of the butyrophilin family proteins, wherein the fragment is capable of binding a gamma delta T cell receptor and is optionally an extracellular domain.

In any of the embodiments disclosed herein, the cancer therapy comprises a heterodimeric protein comprising an alpha chain and a beta chain wherein the alpha chain and the beta chain each independently comprise (a) a first domain comprising a butyrophilin family protein, or fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the first domain comprises two of the same butyrophilin family proteins. In embodiments, the first domain comprises two different butyrophilin family proteins. In embodiments, the butyrophilin family proteins, or a fragment thereof comprise an Ig-like V-type domain. In embodiments, the butyrophilin family proteins, or a fragment thereof are derived from native full length proteins. In embodiments, the first domain comprises one or more fragments of the butyrophilin family proteins, wherein the fragment is capable of binding a gamma delta T cell receptor and is optionally an extracellular domain.

In embodiments, the first domain comprises a polypeptide having an amino acid sequence of: (a) a polypeptide having an amino acid sequence selected from the amino acid sequence of SEQ ID NOs: 24 to 45, or a fragment thereof; and (b) a polypeptide having an amino acid sequence selected from the amino acid sequence of SEQ ID NOs: 24 to 45, or a fragment thereof. In embodiments, the linker comprises a polypeptide having an amino acid sequence selected from the amino acid sequence of SEQ ID NOs: 1-14.

In embodiments, the linker comprises a polypeptide having an amino acid sequence selected from the amino acid sequence of SEQ ID NOs: 15-22.

In embodiments, the targeting domain is an antibody, or antigen binding fragment thereof. In embodiments, the targeting domain is an antibody-like molecule, or antigen binding fragment thereof. In embodiments, the antibody-like molecule is selected from a single-chain antibody (scFv), a single-domain antibody, a recombinant heavy-chain-only antibody (VHH), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2. In embodiments, the antibody-like molecule is an scFv.

In embodiments, the targeting domain is an extracellular domain.

In embodiments, the targeting domain is capable of binding an antigen on the surface of a cancer cell. In embodiments, the targeting domain specifically binds a protein selected from CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDRNEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, CDH18, EPHA3, NY-eso-2, MAGE-A10, MAGE-A3, MAGE-A7, HER3, and GD3. In embodiments, the targeting domain comprises a portion of the extracellular domain of LAG-3, PD-1, TIGIT, CD19, or PSMA. In embodiments, the targeting domain specifically binds CD19. In embodiments, the targeting domain specifically binds PSMA. In embodiments, the targeting domain specifically binds CD33. In embodiments, the targeting domain specifically binds CD20. In embodiments, the targeting domain specifically binds DLL3. In embodiments, the targeting domain specifically binds CLL-1.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linkerforms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally human IgG1. In embodiments, the linker comprises the hinge-CH2-CH3 Fc domain derived from IgG4, optionally human IgG4.

In embodiments, the first and/or second charge polarized core domain further comprise peptides having positively and/or negatively charged amino acid residues at the amino and/or carboxy terminus of the charge polarized core domain.

In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YnXnYnXnYn (where X is a positively charged amino acid such as arginine, histidine or lysine and Y is a spacer amino acid such as serine or glycine, and where each n is independently an integer 0 to 4) (SEQ ID NO: 1), YYnXXnYYnXXnYYn (where X is a positively charged amino acid such as arginine, histidine or lysine and Y is a spacer amino acid such as serine or glycine, and where each n is independently an integer 0 to 4) (SEQ ID NO: 3), and YnXnCYnXnYn (where X is a positively charged amino acid such as arginine, histidine or lysine and Y is a spacer amino acid such as serine or glycine, and where each n is independently an integer 0 to 4) (SEQ ID NO: 5). In embodiments, wherein the peptide comprising positively charged amino acid residues comprises the sequence RKGGKR (SEQ ID NO: 11) or GSGSRKGGKRGS (SEQ ID NO: 12).

In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YnZnYnZnYn (where Z is a negatively charged amino acid such as aspartic acid or glutamic acid and Y is a spacer amino acid such as serine or glycine, and where each n is independently an integer 0 to 4) (SEQ ID NO: 2), YYnZZnYYnZZnYYn (where Z is a negatively charged amino acid such as aspartic acid or glutamic acid and Y is a spacer amino acid such as serine or glycine, and where each n is independently an integer 0 to 4) (SEQ ID NO: 4), and YnZnCYnZnYn (where Z is a negatively charged amino acid such as aspartic acid or glutamic acid and Y is a spacer amino acid such as serine or glycine, and where each n is independently an integer 0 to 4) (SEQ ID NO: 6). In embodiments, the peptide comprising negatively charged amino acid residues comprises the sequence DEGGED (SEQ ID NO: 13) or GSGSDEGGEDGS (SEQ ID NO: 14).

In embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ764. In embodiments, the modulation of a gamma delta T cell is activation of a gamma delta T cell. In embodiments, the heterodimeric protein is capable of forming a synapse between a gamma delta T cell and a tumor cell and/or the heterodimeric protein is capable of contemporaneous activation and targeting of gamma delta T cells to tumor cells.

Combination Therapies and Conjugation

In embodiments, the invention provides for heterodimeric proteins and methods that further comprise administering an additional agent to a subject. In embodiments, the invention pertains to co-administration and/or co-formulation. Any of the compositions described herein may be co-formulated and/or co-administered.

In embodiments, any heterodimeric protein described herein acts synergistically when co-administered with another agent and is administered at doses that are lower than the doses commonly employed when such agents are used as monotherapy. In various embodiments, any agent referenced herein may be used in combination with any of the heterodimeric proteins described herein.

In various embodiments, any of the heterodimeric proteins disclosed herein may be co-administered with another heterodimeric protein disclosed herein. Without wishing to be bound by theory, it is believed that a combined regimen involving the administration of one or more heterodimeric proteins which induce an innate immune response and one or more heterodimeric proteins which induce an adaptive immune response may provide synergistic effects (e.g., synergistic anti-tumor effects).

In various embodiments, any heterodimeric protein which induces an innate immune response may be utilized in the present disclosure. In various embodiments, any heterodimeric protein which induces an adaptive immune response may be utilized in the present disclosure.

In embodiments, inclusive of, without limitation, cancer applications, the present disclosure pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In various embodiments, inclusive of, without limitation, cancer applications, the present additional agent is one or more immune-modulating agents selected from an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), MK-3475 (MERCK), BMS 936559 (BRISTOL MYERS SQUIBB), atezolizumab (TECENTRIQ, GENENTECH), MPDL3280A (ROCHE)), an agent that increases and/or stimulates CD137 (4-1BB) and/or the binding of CD137 (4-1BB) with one or more of 4-1BB ligand (byway of non-limiting example, urelumab (BMS-663513 and anti-4-1BB antibody), and an agent that blocks, reduces and/or inhibits the activity of CTLA-4 and/or the binding of CTLA-4 with one or more of AP2M1, CD80, CD86, SHP-2, and PPP2R5A and/or the binding of OX40 with OX40L (by way of non-limiting example GBR 830 (GLENMARK), MEDI6469 (MEDIMMUNE).

In embodiments, the heterodimeric proteins (and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids. In still other embodiments, the heterodimeric proteins (and/or additional agents) described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The heterodimeric proteins (and/or additional agents) described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

In aspects, the present disclosure relates to a method for treating a melanoma in a human subject comprising a step of administering to the human subject a heterodimeric protein comprising: (a) a first domain comprising one or more butyrophilin family proteins, or fragments thereof capable of binding to a gamma delta (γδ) T cell receptor; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the gamma delta (γδ) T cell receptor comprises a γ chain selected from Vγ9, Vγ4, Vγ2, Vγ8, Vγ3 and Vγ5, and a δ chain selected from Vδ2, Vδ1, Vδ3 and Vδ5. In embodiments, the gamma delta (γδ) T cell receptor is selected from Vg9Vd2 (γ9δ2), Vg9Vd1 (γ9δ1) and Vg9Vd3 (γ9δ3). In embodiments, the fragments of the one or more butyrophilin family proteins comprise extracellular domains and/or variable domains. In embodiments, the heterodimeric protein comprises a portion of BTN2A1 capable of binding a gamma delta (γδ) T cell receptor. In embodiments, the heterodimeric protein comprises the extracellular domain BTN2A1. In embodiments, the heterodimeric protein comprises the variable domain BTN2A1. In embodiments, the heterodimeric protein comprises a portion of BTN3A1 capable of binding a gamma delta (γδ) T cell receptor. In embodiments, the heterodimeric protein comprises the extracellular domain BTN3A1. In embodiments, the heterodimeric protein comprises the variable domain BTN3A1. In embodiments, the heterodimeric protein comprises a portion of BTN2A1 capable of binding a gamma delta (γδ) T cell receptor and/or a portion of BTN3A1 capable of binding a gamma delta (γδ) T cell receptor. In embodiments, the heterodimeric protein comprises the extracellular domain BTN2A1 and/or BTN3A1. In embodiments, the heterodimeric protein comprises the variable domain BTN2A1 and/or BTN3A1.

In aspects, the present disclosure relates to a method for treating a non-small cell lung cancer (NSCLC) in a human subject comprising a step of administering to the human subject a heterodimeric protein comprising: (a) a first domain comprising one or more butyrophilin family proteins, or fragments thereof capable of binding to a gamma delta (γδ) T cell receptor; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the gamma delta (γδ) T cell receptor comprises a γ chain selected from Vγ9, Vγ2, Vγ4, Vγ5, Vγ3 and Vγ8, and a δ chain selected from Vδ1, Vδ2, Vδ3, Vδ8, and Vδ6. In embodiments, the gamma delta (γδ) T cell receptor is selected from Vg9Vd1 (γ9δ1), Vg9Vd2 (γ9δ2), and Vg9Vd3 (γ9δ3). In embodiments, the fragments of the one or more butyrophilin family proteins comprise extracellular domains and/or variable domains. In embodiments, the heterodimeric protein comprises a portion of BTN2A1 capable of binding a gamma delta (γδ) T cell receptor. In embodiments, the heterodimeric protein comprises the extracellular domain BTN2A1. In embodiments, the heterodimeric protein comprises the variable domain BTN2A1. In embodiments, the heterodimeric protein comprises the extracellular domain BTN3A1. In embodiments, the heterodimeric protein comprises the variable domain BTN3A1. In embodiments, the heterodimeric protein comprises a portion of BTN2A1 capable of binding a gamma delta (γδ) T cell receptor and/or a portion of BTN3A1 capable of binding a gamma delta (γδ) T cell receptor. In embodiments, the heterodimeric protein comprises the extracellular domain BTN2A1 and/or BTN3A1. In embodiments, the heterodimeric protein comprises the variable domain BTN2A1 and/or BTN3A1.

In aspects, the present disclosure relates to a method for treating a colorectal cancer in a human subject comprising a step of administering to the human subject a heterodimeric protein comprising: (a) a first domain comprising one or more butyrophilin family proteins, or fragments thereof capable of binding to a gamma delta (γδ) T cell receptor; (b) a second domain comprising a targeting domain, the targeting domain being selected from an (i) antibody, antibody-like molecule, or antigen binding fragment thereof, and (ii) a extracellular domain; and (c) a linker that adjoins the first and second domain. In embodiments, the gamma delta (γδ) T cell receptor comprises a γ chain selected from Vγ9, Vγ4, Vγ2, Vγ5, Vγ3 and Vγ8, and a δ chain selected from Vδ1, Vδ2, Vδ3, and Vδ8. In embodiments, the gamma delta (γδ) T cell receptor is selected from Vg9Vd1 (γ9δ1), Vg9Vd2 (γ9δ2), and Vg9Vd3 (γ9δ3). In embodiments, the fragments of the one or more butyrophilin family proteins comprise extracellular domains and/or variable domains. In embodiments, the heterodimeric protein comprises a portion of BTN2A1 capable of binding a gamma delta (γδ) T cell receptor. In embodiments, the heterodimeric protein comprises the extracellular domain BTN2A1. In embodiments, the heterodimeric protein comprises the variable domain BTN2A1. In embodiments, the heterodimeric protein comprises the extracellular domain BTN3A1. In embodiments, the heterodimeric protein comprises the variable domain BTN3A1. In embodiments, the heterodimeric protein comprises a portion of BTN2A1 capable of binding a gamma delta (γδ) T cell receptor and/or a portion of BTN3A1 capable of binding a gamma delta (γδ) T cell receptor. In embodiments, the heterodimeric protein comprises the extracellular domain BTN2A1 and/or BTN3A1. In embodiments, the heterodimeric protein comprises the variable domain BTN2A1 and/or BTN3A1.

Formulations

In one aspect, the present disclosure provides a pharmaceutical composition, comprising the heterodimeric protein of any of the embodiments disclosed herein.

The heterodimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, any heterodimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are resuspended in a saline buffer (including, without limitation TBS, PBS, and the like).

In various embodiments, the heterodimeric proteins may by conjugated and/or fused with another agent to extend half-life or otherwise improve pharmacodynamic and pharmacokinetic properties. In embodiments, the heterodimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In various embodiments, each of the individual heterodimeric proteins is fused to one or more of the agents described in BioDrugs (2015) 29:215-239, the entire contents of which are hereby incorporated by reference.

Administration, Dosing, and Treatment Regimens

The present disclosure includes the described heterodimeric protein (and/or additional agents) in various formulations. Any heterodimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the heterodimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the heterodimeric protein (and/or additional agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In one embodiment, any heterodimeric protein (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In most instances, administration results in the release of any agent described herein into the bloodstream.

Any heterodimeric protein (and/or additional agents) described herein can be administered orally. Such heterodimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the heterodimeric protein (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In various embodiments, for instance in the treatment of cancer, the heterodimeric protein (and/or additional agents) are administered intratumorally.

In the various embodiments, the present heterodimeric protein allows for a dual effect that provides less side effects than are seen in conventional immunotherapy (e.g., treatments with one or more of OPDIVO, KEYTRUDA, YERVOY, and TECENTRIQ). For example, the present heterodimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any heterodimeric protein (and/or additional agents) described herein as well as the dosing schedule can depend on various parameters, including, but not limited to, the disease being treated, the subject's general health, and the administering physician's discretion. Any heterodimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In various embodiments any heterodimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In various embodiments, the present disclosure relates to the co-administration of a heterodimeric protein which induces an innate immune response and another heterodimeric protein which induces an adaptive immune response. In such embodiments, the heterodimeric protein which induces an innate immune response may be administered before, concurrently with, or subsequent to administration of the heterodimeric protein which induces an adaptive immune response. For example, the heterodimeric proteins may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart. In an illustrative embodiment, the heterodimeric protein which induces an innate immune response and the heterodimeric protein which induces an adaptive response are administered 1 week apart, or administered on alternate weeks (i.e., administration of the heterodimeric protein inducing an innate immune response is followed 1 week later with administration of the heterodimeric protein which induces an adaptive immune response and so forth).

The dosage of any heterodimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any heterodimeric protein (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the heterodimeric protein (and/or additional agents) described herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the heterodimeric protein (and/or additional agents) is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any heterodimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any heterodimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any heterodimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any heterodimeric protein (and/or additional agents) described herein can be administered in a single daily dose, or the total daily dosage can be administered in divided doses of two, three or four times daily. Furthermore, any heterodimeric protein (and/or additional agents) described herein can be administered continuously rather than intermittently throughout the dosage regimen.

Cells and Nucleic Acids

In one aspect, the present disclosure provides an expression vector, comprising a nucleic acid encoding the first and/or second polypeptide chains of the heterodimeric protein of any of any of the embodiments disclosed herein. In embodiments, the expression vector is a mammalian expression vector. In embodiments, the expression vector comprises DNA or RNA. In embodiments, in one aspect, the present disclosure provides a host cell comprising the expression vector of any one of the embodiments disclosed herein.

In various embodiments, the present disclosure provides an expression vector, comprising a nucleic acid encoding the heterodimeric protein (e.g., a heterodimeric protein comprising a first and second polypeptide chains) described herein. In various embodiments, the expression vector comprises DNA or RNA. In various embodiments, the expression vector is a mammalian expression vector.

Both prokaryotic and eukaryotic vectors can be used for expression of the heterodimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λP_{L}. Non-limiting examples of prokaryotic expression vectors may include the λgt vector series such as Agt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the heterodimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In embodiments, expression vectors of the invention comprise a nucleic acid encoding at least the first and/or second polypeptide chains of the heterodimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In an embodiment, the cell is a tumor cell. In another embodiment, the cell is a non-tumor cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In an embodiment, the present disclosure contemplates the use of inducible promoters capable of effecting high level of expression transiently in response to a cue. For example, when in the proximity of a tumor cell, a cell transformed with an expression vector for the heterodimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used fortargeting and/or to facilitate uptake, e.g., capsid proteins or fragments thereof tropic for a particular cell type, antibodies for proteins which undergo internalization in cycling, proteins thattarget intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In one aspect, the invention provides expression vectors for the expression of the heterodimeric proteins (and/or additional agents) that are viral vectors. Many viral vectors useful for gene therapy are known (see, e.g., Lundstrom, Trends Biotechnol., 21: 117, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as α viruses and adenoviruses. Illustrative types of α viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In one embodiment, the invention provides methods of transducing a human cell in vivo, comprising contacting a solid tumor in vivo with a viral vector of the invention.

In various embodiments, the present disclosure provides a host cell, comprising the expression vector comprising the heterodimeric protein described herein.

Expression vectors can be introduced into host cells for producing the present heterodimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E. G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, and cancer patients, private laboratory deposits, public culture collections such as the American Type Culture Collection, or from commercial suppliers.

Cells that can be used for production of the present heterodimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor being treated or prevented, and can be determined by one of skill in the art.

Production and purification of Fc-containing macromolecules (such as Fc fusion proteins) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In various embodiments, production of the heterodimeric proteins contemplated herein may have unique characteristics as compared to traditional Fc containing macromolecules. In certain examples, the heterodimeric proteins may be purified using specific chromatography resins, or using chromatography methods that do not depend upon Protein A capture. In other embodiments, the heterodimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The heterodimeric proteins obtained herein may be additionally ‘polished’ using methods that are specified in the art. In embodiments, the heterodimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In other embodiments, the heterodimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### Definitions

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100.

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present disclosure, the preferred materials and methods are described herein. It is to be understood that the terminology used herein is for the purpose of describing particular embodiments only, and is not intended to be limiting.

Any aspect or embodiment described herein can be combined with any other aspect or embodiment as disclosed herein.

The invention will be further described in the following examples, which do not limit the scope of the invention described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1: Gamma and Delta Chain Pairing in Matched Peripheral Blood Mononuclear Cells (PBMCs) and Dissociated Tumor Tissue from Cancer Patient

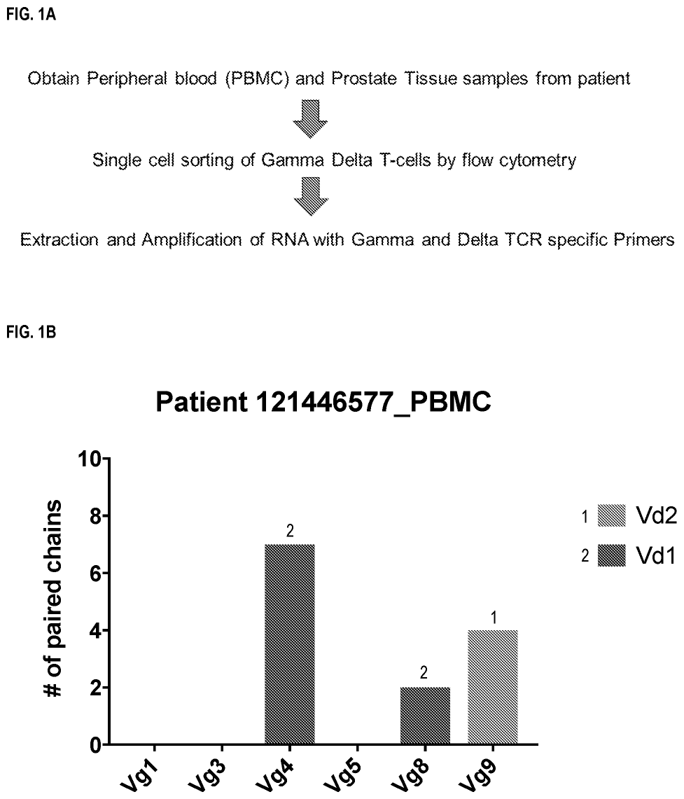

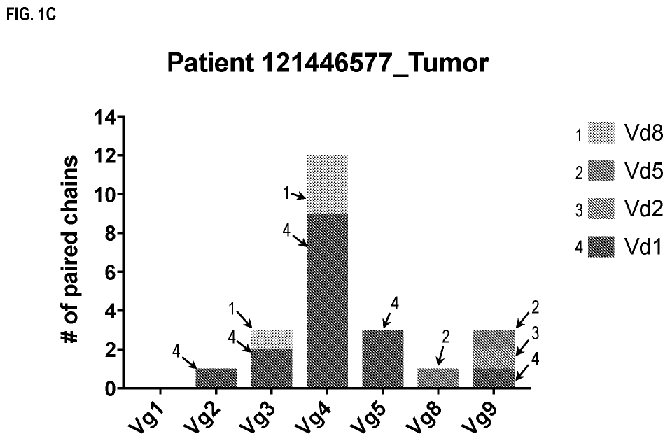

To compare the gamma and delta chain pairing in matched peripheral blood mononuclear cells (PBMCs) and dissociated tumor tissue from a prostate cancer patient, sequencing of gamma and delta chain was performed. As shown in FIG. 1A, PBMCs and dissociated tumor tissue were obtained from a prostate adenocarcinoma patient. Gamma delta T cells (γδ T cells) in the samples were single cell sorted into 96 well plates by flow cytometry following antibody staining of the gamma delta TCR. RNA was prepared and the gamma and delta chains were sequenced using the iPAIR PCR technique (iRepertoire). The iPair analyzer software was used to identify gamma and delta chain sequences and pairings on a single cell level. The observed number of delta chain was plotted against the paired gamma chain. As shown in FIG. 1B, the patient's PBMCs showed limited diversity of specific gamma-delta pairing. In contrast, as shown in FIG. 1C, gamma and delta chain pairings in the patient's dissociated tumor samples showed more diversity.

The data indicate that in the patient PBMCs there was a higher prevalence of Vγ9δ2 and Vγ4δ1 TCR expressing GDT cells. Vγ8δ1 TCR expressing GDT cells were also detected at a lower abundance. In the dissociated tumor sample a higher diversity of gamma delta chain pairing in GDT cells was observed with the Vγ4δ1 being the most abundant. Accordingly, a treatment directed at Vγ4δ1 is likely to be more effective against the tumor in the patient compared to other γδ TCRs. Moreover, these data suggested that distinct BTN heterodimers may preferentially activate tissue-restricted subsets of γδ T cells.

### Example 2: Diversity of Antigen Specificity Gamma and Delta Chain in Matched Peripheral Blood Mononuclear Cells (PBMCs) and Dissociated Tumor Tissue from a Prostate Cancer Patient

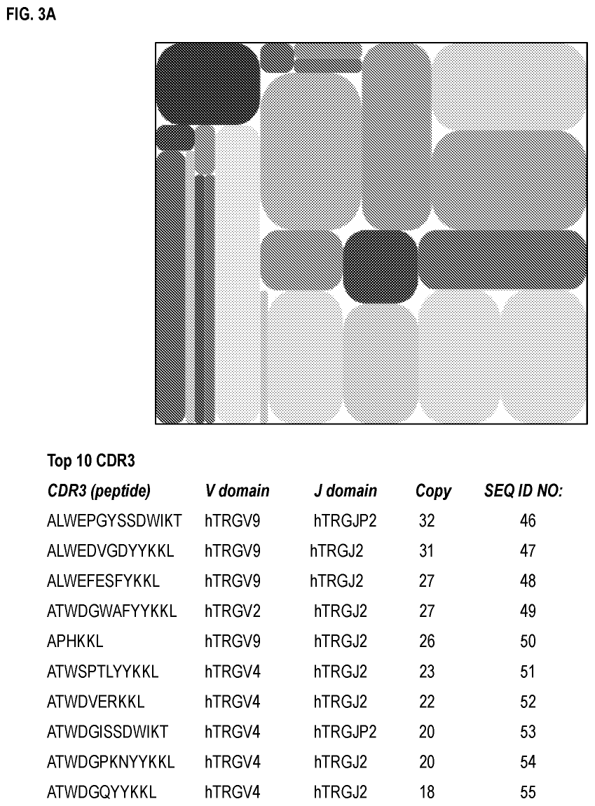

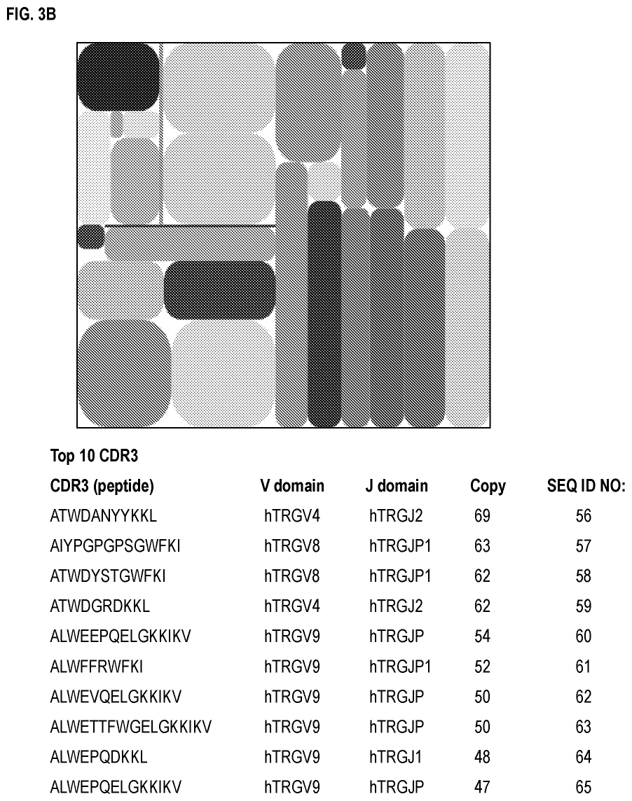

To understand whether the observed a higher diversity of gamma delta chain pairing in tumor correlates with any alterations in antigen specificity, the gamma chain usage and the sequences of gamma chain hypervariable or complementarity-determining region CDR3 were compared. As shown in FIG. 3A, the dissociated tumor exhibited some diversity of the gamma chain CDR3 sequences. The sequences of the top 10 CDR3 are listed below the CDR3 tree map in FIG. 3A. In comparison, the diversity of the CDR3 sequences in PBMCs also showed diversity of CDR usage (FIG. 3B). The sequences of the top 10 CDR3 are listed below the CDR3 tree map in FIG. 3A-FIG. 3B. The comparison of usage of gamma chain again confirmed higher diversity of the gamma chain usage in the tumor.

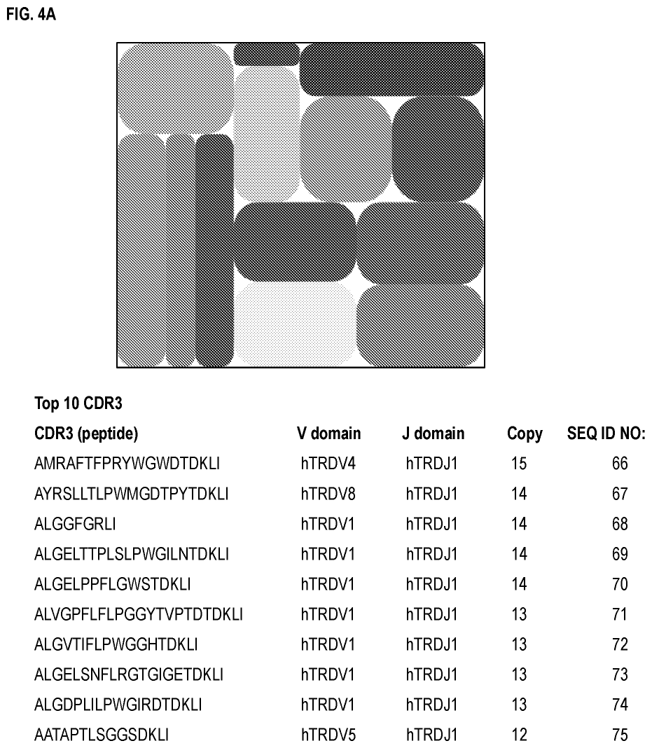

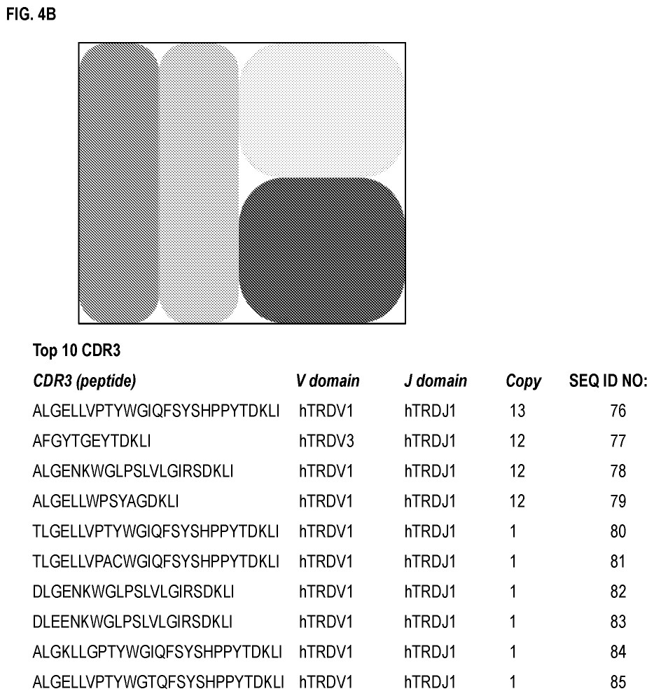

The CDR3 tree map comparison of delta chain usage in showed similar trend tumor compared to PBMC. For example, both the dissociate tumor (FIG. 4A) and PBMCs (FIG. 4B) showed the diversity of the delta chain CDR3 sequences. The sequences of the top 10 CDR3 are listed below the CDR3 tree map in FIG. 4A-FIG. 4B. The comparison of usage of gamma chain again confirmed higher diversity of the delta chain usage in the tumor.

The data indicate that a treatment directed at specific γδ TCRs would target the treatment to the patient's tumor. Accordingly, the method of analyzing γδ TCR repertoire in the tumor, in comparison with peripheral blood is useful in a method for identifying a cancer therapy, or method of selecting a patient in the therapy.

### Example 3: Single Cell Sorting and Sequencing of γδ TCRs from Dissociated Tumors of Select TCGA Tumor Types

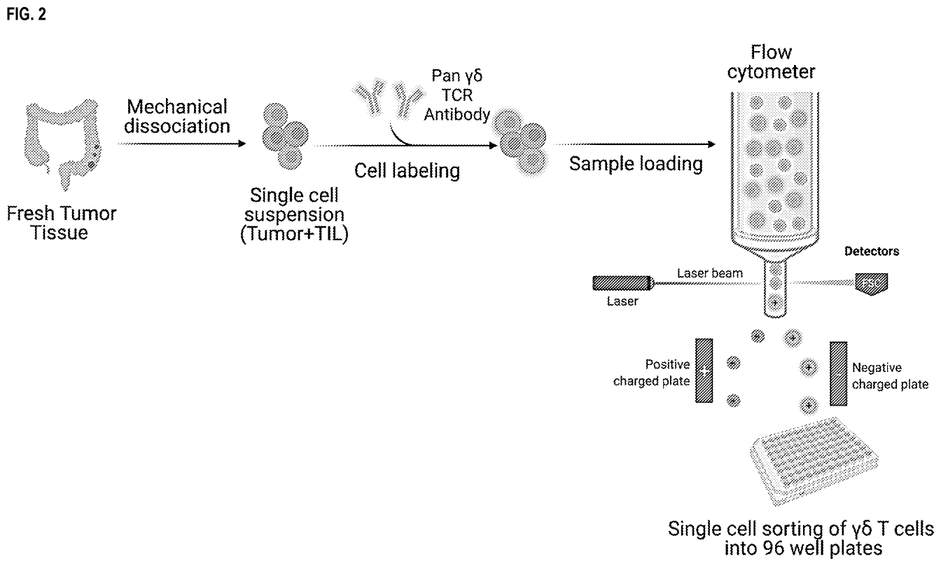

To extend this analysis further, single cell sequencing analysis was performed with samples from cancer patients to determine gamma and delta chain pairing in tumor tissue and tumor infiltrating lymphocytes (TIL) as shown in FIG. 2. Briefly, freshly resected tumor tissue was mechanically dissociated to create a single cell suspension of tumor and tumor infiltrating lymphocytes (TIL). Gamma delta T cells in the samples were stained with a pan γδ TCR antibody and single cell sorted into 96 well plates by flow cytometry. The RNA was extracted from the cells and TCRγ and TCRδ chains were amplified and sequenced using the iPAIR PCR technique (iRepertoire). The iPair analyzer software was used to identify gamma and delta chain sequences and pairings on a single cell level.

To characterize potential differences between peripheral blood and tissue-restricted γδ T cells, a multi-layered analysis was performed, including single-cell sequencing of γδ T TCR and CDR3 analysis from paired peripheral blood and tumor tissues from human cancer patients with melanoma, prostate, and colon cancer. Bolotin et al., TCR identification was assessed using MiXCR version 2.1.6 with custom R scripts. MiXCR: software for comprehensive adaptive immunity profiling, Nature Methods 12: 380-381(2015). IMGT TCR repertoire sequence reference imgt.201918-4.sv5.json was used to facilitate alignment of germline TCRγ and TCRδ genes and assembly of repertoires. Lefranc, IMGT, the International ImMunoGeneTics Information System, Cold Spring Harb Protoc 2011(6):595-603 (2011). Bulk RNA-seq fastq files for four of the cancer genome atlas (TCGA) tumor types (COAD, DLBC, SARC and STAD) were used as input. To maximize sensitivity of capturing TCRγ and TCRδ alignments, the align function in MiXCR was run with the assemblePartial and extend options. Quantification of gene expression was generated using Salmon's quasi-alignment method. Patro et al., Salmon provides fast and bias-aware quantification of transcript expression, Nature Methods 14: 417-419 (2017). Gencode GrCH38 v27 CHR transcripts were used to build the index used for Salmon. Frankish et al., “GENCODE reference annotation for the human and mouse genomes.” Nucleic Acids Research 47 (D1): D766-D773 (2018).

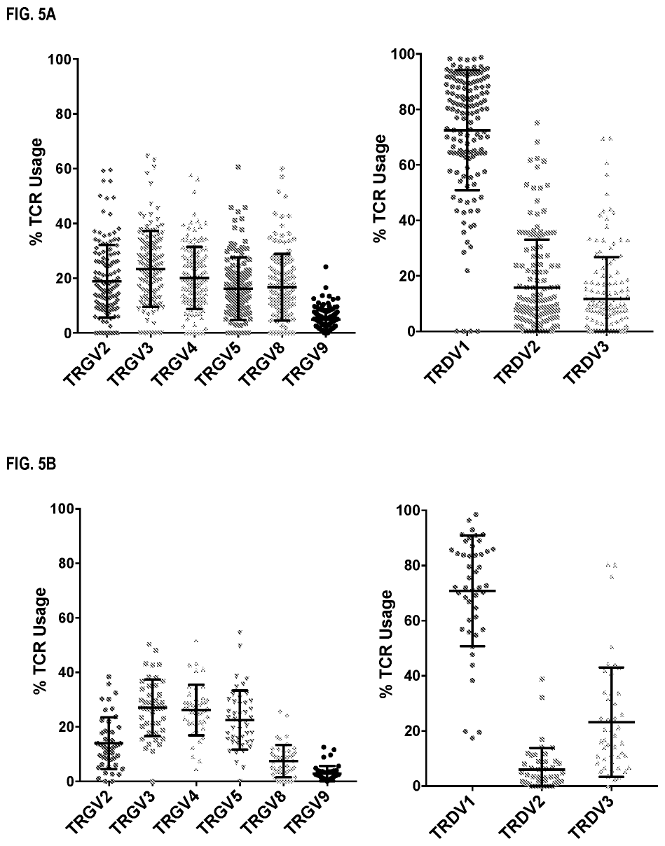

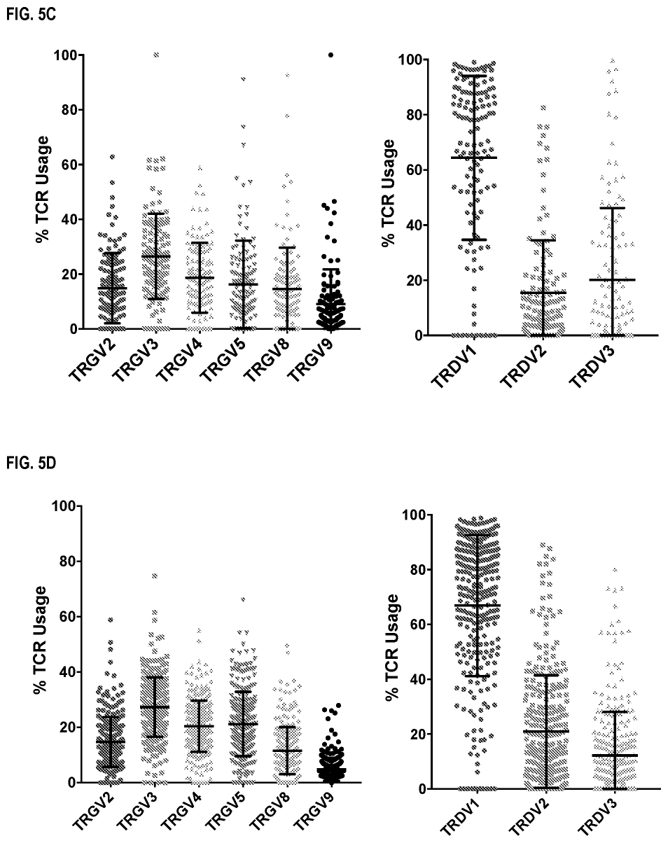

The TCGA data sets for colorectal adeno-carcinoma (FIG. 5A), diffuse large B cell lymphoma (DLBCL) (FIG. 5B), Sarcoma (FIG. 5C), and stomach adeno-carcinoma (FIG. 5D) were analyzed for γδ TCR usage using the MiXCR framework. Results revealed that all tumors expressed multiple Vγ chains with broad usage while Vδ chains were restricted to TRDV1, 2 and 3 with Vδ1 being the most prevalent across the four tumor types.

### Example 4: γδ TCR Usage and CDR3 Diversity in Matched Tumor and Peripheral Blood in Colorectal Adenocarcinoma Patients Detected by Single Cell RNA Sequencing

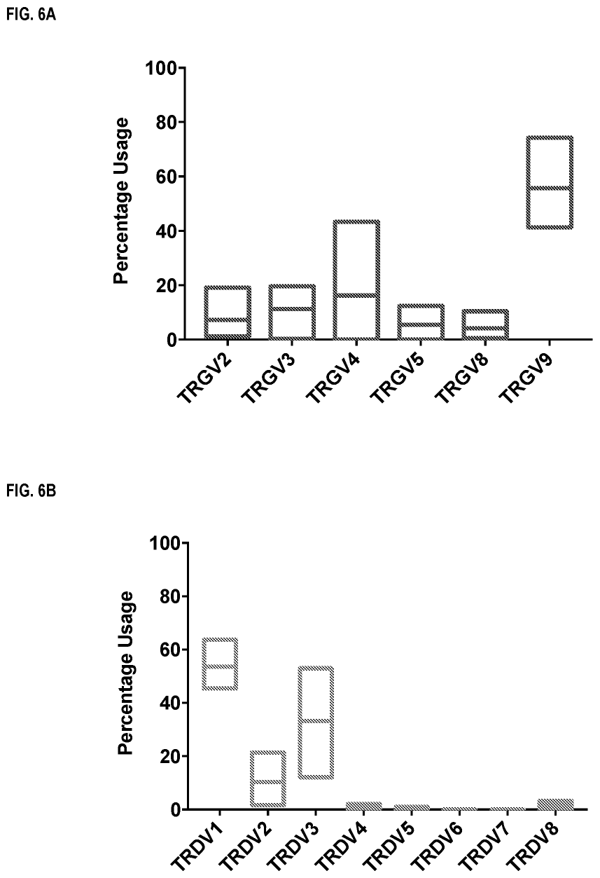

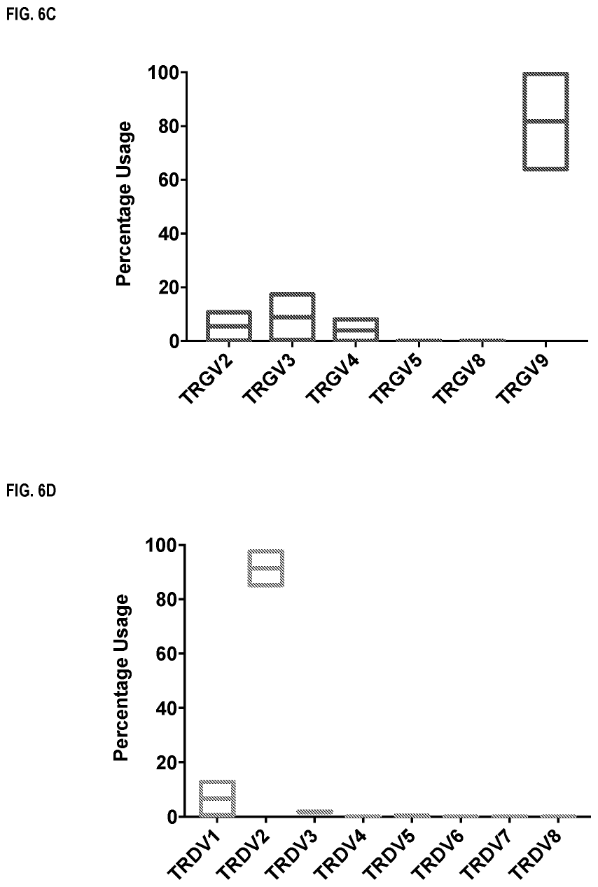

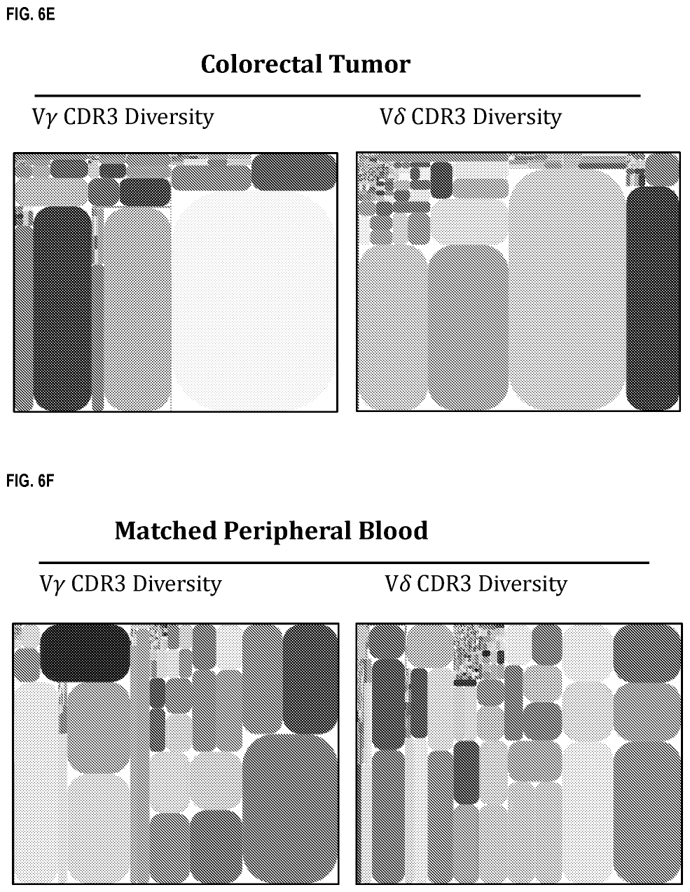

Next, single cell RNA sequencing of γδ TCRs was performed from colorectal tumors and matched peripheral blood. FIG. 6A shows the single cell RNA sequencing of Vγ chain from colorectal tumors. FIG. 6B shows the single cell RNA sequencing of Vδ chain from colorectal tumors. FIG. 6C shows the single cell RNA sequencing of Vγ chain from matched peripheral blood. FIG. 6D shows the single cell RNA sequencing of Vδ chain from matched peripheral blood. As shown in FIG. 6A to FIG. 6D, a distinct difference in usage of Vγ chains and Vδ chains was observed between tumors and matched peripheral blood. Tumor-derived γδ T cells expressed multiple γ chains, where TRGV9 and TRGV4 were most abundant (FIG. 6A). Colorectal tumor derived γδ T cells also expressed more TRDV1 compared to peripheral blood γδ T cells, which predominantly expressed TRDV2 (compare FIG. 6B and FIG. 6D).

A sequence analysis of CDR3 of the Vγ and Vδ chains in peripheral blood and the tumor was performed and depicted in form of representative CDR3 tree maps from a single patient. FIG. 6E shows the CDR3 sequence analysis of the Vγ and Vδ chains in colorectal cancer tumor. FIG. 6F shows the CDR3 sequence analysis of the Vγ and Vδ chains in peripheral blood. As shown in FIG. 6E and FIG. 6F, a greater diversity in both the γ and δ chains was observed in the peripheral blood compared to the tumor. Clonal expansion of γδ T cells was observed in colorectal tumors when compared to peripheral blood.

### Example 5: Correlation of BTN/L and γδ TCR Expression in Colorectal Adenocarcinoma

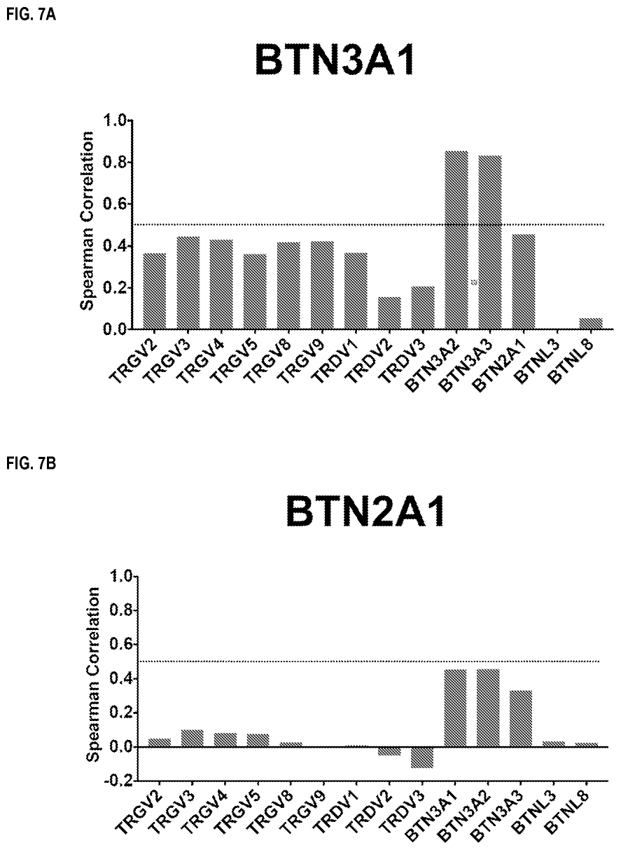

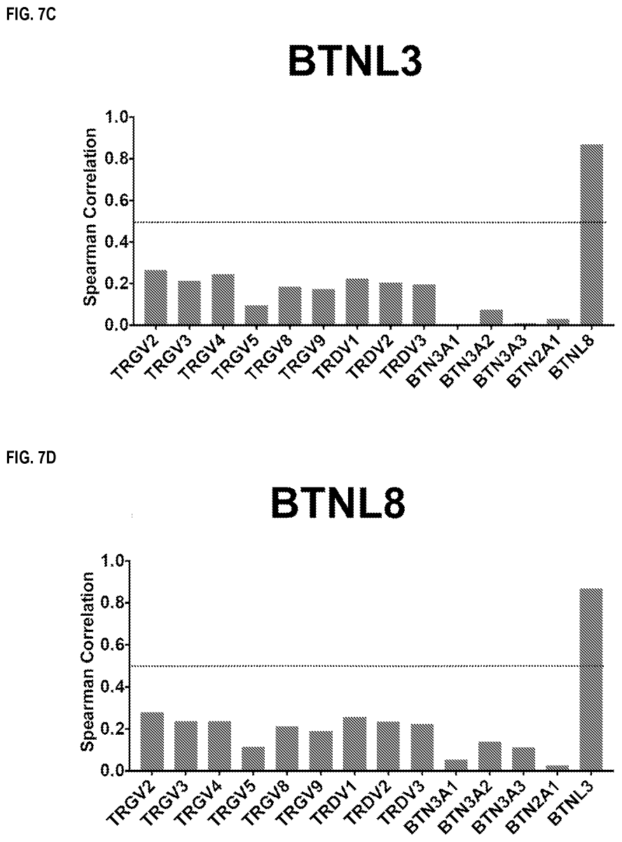

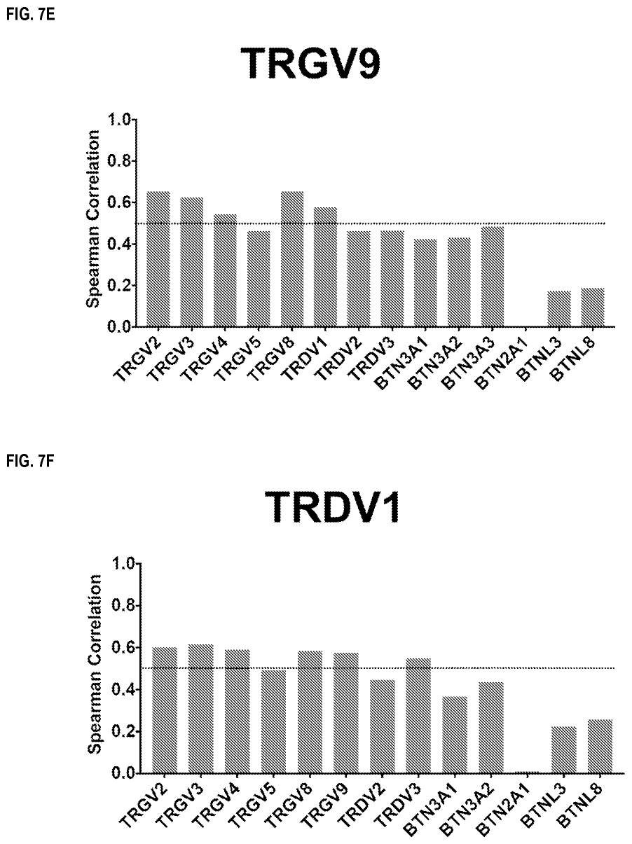

Next, to evaluate the correlation of BTN/L and γδ TCR expression, Spearman correlation analysis was performed on the expression of certain BTN or BTNL proteins in colorectal adenocarcinoma derived γδ T cells through single cell RNA sequencing. The results are shown for BTN3A1 (FIG. 7A), BTN2A1 (FIG. 7B), BTNL3 (FIG. 7C) and BTNL8 (FIG. 7D). Expression of gamma chain (TRGV9) (FIG. 7E) and delta chain (TRDV1) (FIG. 7F) was also similarly analyzed. While BTN3A1 expression partially correlated with TRGV9 and TRDV1, there was a distinct lack of correlation with BTN2A1 expression with these select gamma and delta chains.

These results demonstrate, interalia, that butyrophilin expression and γδ TCR expression correlate with each other. Accordingly, these results demonstrate that evaluating the sample for the presence, absence, or level of one or more gamma/delta T-cell receptor (TCR) chains and/or butyrophilin proteins may be used for identifying a cancer therapy, selecting a patient for a cancer treatment and/or for making an agent for the treatment of a cancer in a cancer patient.

### Example 6: Development of a Jurkat-76 Cell Line-Based In Vitro Assay to Screen BTN/L Pairs that Activate Specific γδ TCRs Detected Using Single Cell RNA Sequencing

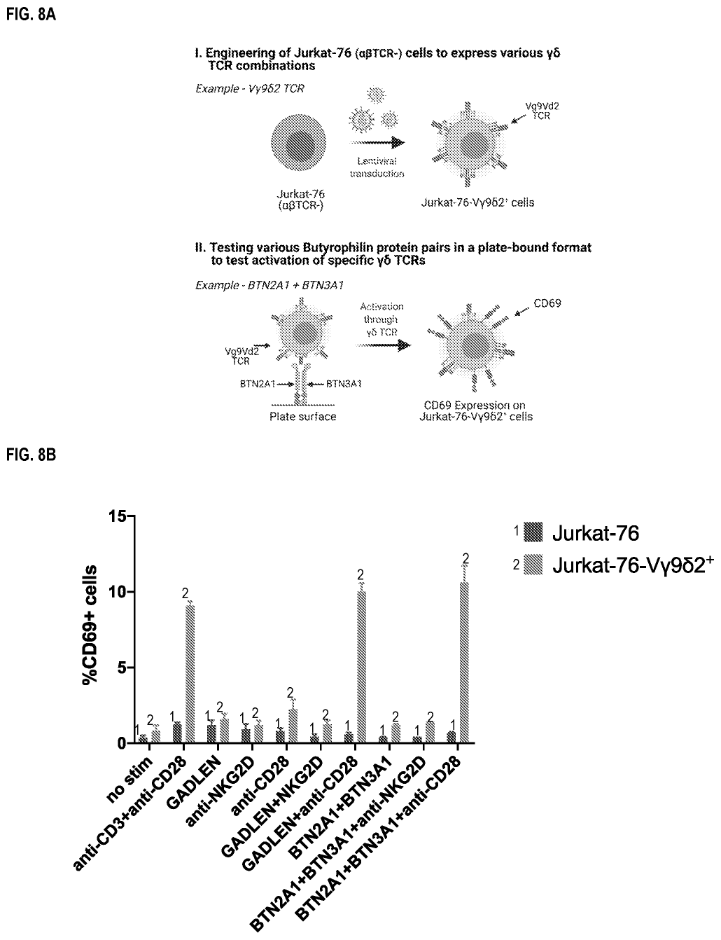

To study whether BTN/L pairs that activate specific γδ TCRs, an in vitro assay was developed. Briefly, a Jurkat-76 cell line-based reporter system was developed to screen various BTN/L combinations that can activate specific γδ TCRs that are uncovered by single cell RNA sequencing pipeline. In an illustrative example, as shown in FIG. 8A (top panel), Jurkat 76 cells expressing γ9δ2 T cell receptor (TCR) was constructed. As illustrated in FIG. 8A (bottom panel), expression of CD69 may be used as a marker of γδ T cell activation. To test this, parental Jurkat-76 or Jurkat-76 expressing Vγ9δ2 TCR complex were stimulated with plate-bound agent(s) as indicated along the X-axis for 24 hours. Cell activation was assessed by surface expression of CD69 as determined by flow cytometry. As shown in FIG. 8B, shows plate-bound BTN2A1 plus BTN3A1 were able to activate the Vγ9δ2 TCR in the presence of CD28 costimulation. Consistent with the role of GADLEN (or BTN2A1+3A1) and co-stimulation in activating primary gamma/delta T cells, GADLEN or BTN2A1+BTN3A1 combination with anti-CD28 stimulation resulted in Jurkat-76-Vg9d2+ activation. Unlike primary γδ T cells, Jurkat-76-Vγ9δ2+ did not express NKG2D. Therefore, GADLEN or BTN2A1+BTN3A1 in combination with anti-NKG2D did not result in cell activation in this assay format. GADLEN or BTN2A1+BTN3A1 mediated activation of Jurkat-76 cells only occurred when Vγ9δ2 TCR was present, further confirming direct interaction of BTN2A1/3A1 with TCR.

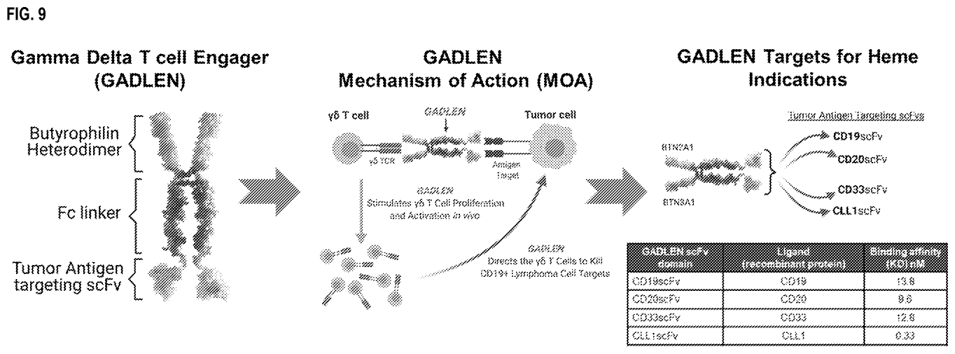

The non-limiting GADLEN design shown in FIG. 9 consists of a heterodimer of butyrophilin extracellular domains fused to a tumor antigen targeting scFv domain via an Fc linker. Without being bound by theory, the GADLENs activate the γδ TCR through the BTN/L heterodimer domain and induce targeted killing of a tumor cell via the binding of a surface tumor antigen by the scFv domain (FIG. 9). Therefore, as shown in FIG. 9, without being bound by theory, GADLEN therapeutics that activate of the γδ TCR through the BTN/L heterodimer domains and cause targeted killing of tumor cells may be generated.

### Example 7: Gamma (TRGV) and Delta (TRDV) Chain T-Cell Receptor Usage in Melanoma, Non-Small Cell Lung and Colorectal Cancer Tumors

Forty-six tumors (twenty colorectal cancer tumors, twenty non-small cell lung cancer (NSCLC) and six melanoma tumors were subjected to the analysis of gamma-delta (γδ) chain usage. Single cell sequencing analysis as well as bulk RNA sequencing of gamma (TRGV) and delta (TRDV) chains was performed on T-cells in patient tumor samples.

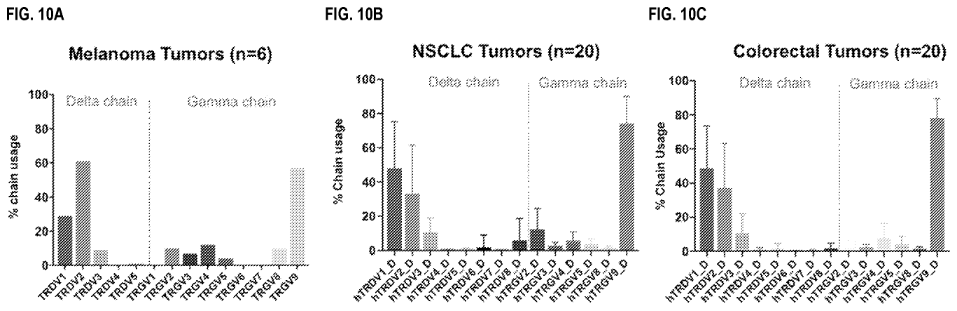

For single cell sequencing analysis, briefly, freshly resected tumor tissue was mechanically dissociated to create a single cell suspension of tumor and tumor infiltrating lymphocytes (TIL). Gamma delta T cells in the samples were stained with a pan γδ TCR antibody and single cell sorted into 96 well plates by flow cytometry. The RNA was extracted from the cells and TCRγ and TCRδ chains were amplified and sequenced using the PAIR PCR technique (iRepertoire). The Pair analyzer software was used to identify gamma and delta chain sequences and pairings on a single cell level. In addition, the TCRγ and TCRδ chains in tumor-infiltrating immune cells were characterized from bulk tumor RNA-seq data. The usage of gamma (TRGV) and delta (TRDV) chain T-cell receptor as obtained from these data are shown in FIG. 10A to FIG. 10C.

As shown in FIG. 10A, TRGV9 was the most commonly used gamma chain in melanoma tumors, which accounted for over 50% usage. TRGV4, TRGV2, TRGV8, TRGV3, and TRGVδ expression was also detected in melanoma tumors (FIG. 10A). TRDV2 accounted for over 60% of gamma chain usage in melanoma tumors, with TRDV1 (over 25%) and TRDV3 (about 10%) showing high usage (FIG. 10A). TRDVδ also showed detectable usage in melanoma tumors (FIG. 10A). These data indicate, inter alia, that Vg9Vd2 (γ9δ2), Vg9Vd1 (γ9δ1) and Vg9Vd3 (γ9δ3) are most common γδ receptors in melanoma.

As shown in FIG. 10B, TRGV9 was the most commonly used gamma chain in NSCLC tumors, which accounted for about 75% usage. TRGV2, TRGV4, TRGV5, TRGV3, and TRGV8 expression was also detected in NSCLC tumors (FIG. 10B). TRDV1 accounted for about 50% of gamma chain usage in NSCLC tumors, with TRDV2 (over 30%) and TRDV3 (about 10%) showing high usage (FIG. 10B). TRDV8, TRDV6, TRDV5, TRDV4, and TRDV7 also showed detectable usage in NSCLC tumors (FIG. 10B). These data indicate, inter alia, that Vg9Vd1 (γ9δ1), Vg9Vd2 (γ9δ2), and Vg9Vd3 (γ9δ3) are most common γδ receptors in NSCLC.

As shown in FIG. 10C, TRGV9 was the most commonly used gamma chain in colorectal tumors, which accounted for over 75% usage. TRGV4, TRGV2, TRGVδ, TRGV3, and TRGV8 expression was also detected in colorectal tumors (FIG. 10C). TRDV1 accounted for about 50% of gamma chain usage in colorectal tumors, with TRDV2 (about 35%) and TRDV3 (about 10%) showing high usage (FIG. 10C). TRDV8, TRDV5, TRDV4, TRDV6, and TRDV7 also showed detectable usage in colorectal tumors (FIG. 10C). These data indicate, inter alia, that Vg9Vd1 (γ9δ1), Vg9Vd2 (γ9δ2), and Vg9Vd3 (γ9δ3) are most common γδ receptors in colorectal cancers.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for identifying a cancer therapy, the method comprising:
(a) obtaining a biological sample, the sample comprising one or more gamma/delta T cells;
(b) evaluating the sample for the presence, absence, or level of one or more gamma/delta T-cell receptor (TCR) chains; and
(c) selecting a cancer therapy having an ability to signal at the gamma/delta TCR, the cancer therapy comprising one or more butyrophilin proteins, or a fragment thereof.

2. A method for selecting a patient for a cancer treatment, the method comprising:
(a) obtaining a biological sample, the sample comprising one or more gamma/delta T cells;
(b) evaluating the sample for the presence, absence, or level of one or more gamma/delta T-cell receptor (TCR) chains; and
(C) selecting a cancer therapy having an ability to signal at the gamma/delta TCR, the cancer therapy comprising one or more butyrophilin proteins, or a fragment thereof.

3. A method for making an agent for the treatment of a cancer in a cancer patient, comprising
(a) obtaining the agent for the treatment of a cancer, the obtaining comprising:
(i) evaluating the biological sample from a cancer patient for the presence, absence, or level of one or more gamma/delta T-cell receptor (TCR) chains; and
(ii) selecting a cancer therapy having an ability to signal at the one or more gamma/delta T-cell receptor (TCR) chains, the cancer therapy comprising one or more butyrophilin proteins, or a fragment thereof;
(b) formulating the identified agent for administration to a cancer patient.

4. The method of any one of claims 1 to 3, wherein the evaluating comprises measuring the presence, absence, or level of one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

5. A method for identifying a cancer therapy for a tumor, the method comprising:
(a) obtaining a biological sample, the sample comprising one or more gamma/delta T cells;
(b) evaluating the sample for the presence, absence, or level of one or more butyrophilin proteins; and
(c) selecting a cancer therapy having an ability to signal at a gamma/delta TCR, the cancer therapy comprising the one or more butyrophilin proteins, or a fragment thereof.

6. A method for selecting a patient for a cancer treatment, the method comprising:
(a) obtaining a biological sample, the sample comprising one or more gamma/delta T cells;
(b) evaluating the sample for the presence, absence, or level of one or more butyrophilin proteins; and
(c) selecting a cancer therapy having an ability to signal at a gamma/delta TCR, the cancer therapy comprising the one or more butyrophilin proteins, or a fragment thereof.

7. The method of claim 5 or claim 6, wherein the evaluating comprises measuring the presence, absence, or level of one or more butyrophilin proteins selected from BTN1A1, BTN2A1, BTN2A2, BTN2A3, BTN3A1, BTN3A2, BTN3A3, BTNL2, BTNL3, BTNL8, BTNL9, BTNL10, and SKINTL.

8. The method of any one of claims 5 to 7, wherein the evaluating comprises measuring the presence, absence, or level of one or more butyrophilin proteins selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

9. The method of any one of claims 1 to 8, wherein the biological sample is a tissue sample.

10. The method of claim 9, wherein the tissue sample is selected from fresh tissue sample, frozen tumor tissue specimen, cultured cells, circulating tumor cells, and a formalin-fixed paraffin-embedded tumor tissue specimen.

11. The method of claim 9 or claim 10, wherein the tissue sample is a biopsy sample.

12. The method of claim 11, wherein the biopsy sample is selected from endoscopic biopsy, bone marrow biopsy, endoscopic biopsy (e.g. cystoscopy, bronchoscopy and colonoscopy), needle biopsy (e.g. fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g. shave biopsy, punch biopsy, and incisional biopsy) and surgical biopsy.

13. The method of any one of claims 9 to 12, wherein the tissue sample is selected from bone, bone marrow, lung, brain, liver, adrenal gland, colon, intestine, esophagus, pancreas, urinary bladder, breast, lymph node, and skin.

14. The method of any one of claims 1 to 8, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

15. The method of any one of claims 9 to 14, wherein the biological sample is obtained by a technique selected from scrapes, swabs, and biopsy.

16. The method of any one of claims 9 to 15, wherein the biological sample is obtained by use of brushes, (cotton) swabs, spatula, rinse/wash fluids, punch biopsy devices, puncture of cavities with needles or surgical instrumentation.

17. The method of any one of claims 1 to 16, wherein the biological sample is a tumor sample.

18. The method of claim 17, wherein the tumor is metastatic.

19. The method of claim 17 or claim 18, wherein the tumor has metastasized to a tissue or an organ.

20. The method of claim 19, wherein the tissue or the organ is selected from bone, bone marrow, lung, brain, liver, adrenal gland, colon, intestine, esophagus, pancreas, urinary bladder, breast, lymph node, and skin.

21. The method of any one of claims 17 to 20, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In some embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), Meigs' syndrome cancer; renal carcinoma; colorectal cancer; and adrenal cancer.

22. The method of any one of claims 1 to 21, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

23. The method of any one of claims 5 to 22, wherein the evaluating is performed by contacting the sample with an agent that specifically binds to one or more butyrophilin selected from BTN1A1, BTN2A1, BTN2A2, BTN2A3, BTN3A1, BTN3A2, BTN3A3, BTNL2, BTNL3, BTNL8, BTNL9, BTNL10, and SKINTL.

24. The method of claim 23, wherein the evaluating is performed by contacting the sample with an agent that specifically binds to one or more butyrophilin selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

25. The method of claim 23 or claim 24, wherein the agent that specifically binds to one or more butyrophilin is an antibody or fragment thereof.

26. The method of any one of claims 1 to 4 or 9 to 22, wherein the evaluating is performed by contacting the sample with an agent that specifically binds to one or more gamma/delta T-cell receptor (TCR) chains selected from Vγ2, Vγ3, Vγ4, Vγ5, Vγ8, Vγ9, Vγ11, Vδ1, Vδ2, Vδ3, Vδ4, Vδ5, Vδ6, Vδ7, and Vδ8.

27. The method of claim 26, wherein the agent that specifically binds to one or more TCR is an antibody or fragment thereof.

28. The method of claim 27, wherein the antibody is a recombinant antibody, a monoclonal antibody, a polyclonal antibody, or fragment thereof.

29. The method of any one of claims 5 to 22, wherein the level and/or activity of one or more butyrophilin is measured by contacting the sample with an agent that specifically binds to one or more of the nucleic acids.

30. The method of any one of claims 1 to 4 or 9 to 22, wherein the level and/or activity of one or more TCR is measured by contacting the sample with an agent that specifically binds to one or more of the nucleic acids.

31. The method of claim 29 or claim 30, wherein the agent that specifically binds to one or more of the nucleic acids is a nucleic acid primer or probe.

32. The method of any one of claims 1 to 4, or 9 to 22, or 26 to 28, or 30, or 31, wherein, when the presence, or higher level of:
TCR Vγ9δ2 is detected in tumor compared to healthy tissue or sample from an healthy individual, the cancer therapy comprising human BTN2A1, BTN3A1, BTN2A2 and/or BTN2A3 butyrophilin protein, or a fragment thereof is selected; and/or
TCR Vδ4 is detected in tumor compared to healthy tissue or sample from an healthy individual, the cancer therapy comprising human BTNL3 butyrophilin protein, or a fragment thereof is selected.

33. The method of any one of claims 5 to 25, 29, or 31, wherein, when presence, or higher level of:
human BTN1A1 is detected in tumor compared to healthy tissue or sample from an healthy individual, the cancer therapy comprising human BTN1A1 butyrophilin protein, or a fragment thereof is selected;
human BTNL2 is detected in tumor compared to healthy tissue or sample from an healthy individual, the cancer therapy comprising human BTNL2 butyrophilin protein, or a fragment thereof is selected;
human BTN2A1 is detected in tumor compared to healthy tissue or sample from an healthy individual, the cancer therapy comprising human BTN2A1 butyrophilin protein, or a fragment thereof is selected;
human BTN2A2 is detected in tumor compared to healthy tissue or sample from an healthy individual, the cancer therapy comprising human BTN2A2 butyrophilin protein, or a fragment thereof is selected;
human BTN2A3 is detected in tumor compared to healthy tissue or sample from an healthy individual, the cancer therapy comprising human BTN2A3 butyrophilin protein, or a fragment thereof is selected;
human BTN2A3 is detected in tumor compared to healthy tissue or sample from an healthy individual, the cancer therapy comprising human BTN2A3 butyrophilin protein, or a fragment thereof is selected;
human BTN3A1 is detected in tumor compared to healthy tissue or sample from an healthy individual, the cancer therapy comprising human BTN3A1 butyrophilin protein, or a fragment thereof is selected;
human BTN3A2 is detected in tumor compared to healthy tissue or sample from an healthy individual, the cancer therapy comprising human BTN3A2 butyrophilin protein, or a fragment thereof is selected;
human BTN3A3 is detected in tumor compared to healthy tissue or sample from an healthy individual, the cancer therapy comprising human BTN3A3 butyrophilin protein, or a fragment thereof is selected;
human BTNL8 is detected in tumor compared to healthy tissue or sample from an healthy individual, the cancer therapy comprising human BTNL8 butyrophilin protein, or a fragment thereof is selected;
human BTNL9 is detected in tumor compared to healthy tissue or sample from an healthy individual, the cancer therapy comprising human BTNL9 butyrophilin protein, or a fragment thereof is selected;
human BTNL10 is detected in tumor compared to healthy tissue or sample from an healthy individual, the cancer therapy comprising human BTNL10 butyrophilin protein, or a fragment thereof is selected; and/or
human SKINTL is detected in tumor compared to healthy tissue or sample from an healthy individual, the cancer therapy comprising human SKINTL butyrophilin protein, or a fragment thereof is selected.

34. The method of any one of claims 1 to 33, wherein the cancer therapy comprises a heterodimeric protein comprising:
(a) a first domain comprising one or more butyrophilin family proteins, or a fragment thereof;
(b) a second domain comprising a targeting domain, the targeting domain being selected from an (i) antibody, antibody-like molecule, or antigen binding fragment thereof, and (ii) a extracellular domain; and
(c) a linker that adjoins the first and second domain.

35. The method of any one of claims 1 to 34, wherein the cancer therapy comprises a heterodimeric protein comprising an alpha chain and a beta chain wherein the alpha chain and the beta chain each independently comprise
(a) a first domain comprising a butyrophilin family protein, or fragment thereof;
(b) a second domain comprising a targeting domain, the targeting domain being selected from an (i) antibody, antibody-like molecule, or antigen binding fragment thereof, and (ii) a extracellular domain; and
(c) a linker that adjoins the first and second domain.

36. The method of claim 34 or claim 35, wherein the first domain comprises two of the same butyrophilin family proteins.

37. The method of claim 34 or claim 35, wherein the first domain comprises two different butyrophilin family proteins.

38. The method of any one of claims 34 to 37, wherein the butyrophilin family proteins, or a fragment thereof comprises an Ig-like V-type domain.

39. The method of any one of claims 34 to 37, wherein the butyrophilin family proteins, or a fragment thereof are derived from native full length proteins.

40. The method of any one of claims 34 to 39, wherein the first domain comprises one or more fragments of the butyrophilin family proteins, wherein the fragment is capable of binding a gamma delta T cell receptor and is optionally an extracellular domain.

41. The method of any one of claims 34 to 40, wherein the targeting domain is an antibody, or antigen binding fragment thereof.

42. The method of any one of claims 34 to 40, wherein the targeting domain is an antibody-like molecule, or antigen binding fragment thereof.

43. The method of claim 42, wherein the antibody-like molecule is selected from a single-chain antibody (scFv), a single-domain antibody, a recombinant heavy-chain-only antibody (VHH), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2.

44. The method of claim 42 or claim 43, wherein the antibody-like molecule is an scFv.

45. The method of any one of claims 34 to 40, wherein the targeting domain is an extracellular domain.

46. The method of any one of claims 34 to 45, wherein the targeting domain is capable of binding an antigen on the surface of a cancer cell.

47. The method of any one of claims 34 to 46, wherein the targeting domain specifically binds a protein selected from CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, CDH18, EPHA3, NY-eso-2, MAGE-A10, MAGE-A3, MAGE-A7, HER3, and GD3.

48. The method of any one of claims 34 to 47, wherein the targeting domain comprises a portion of the extracellular domain of LAG-3, PD-1, TIGIT, CD19, or PSMA.

49. The method of any one of claims 34 to 48, wherein the targeting domain specifically binds CD19.

50. The method of any one of claims 34 to 48, wherein the targeting domain specifically binds PSMA.

51. The method of any one of claims 34 to 50, wherein the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus.

52. The method of claim 51, wherein the linker forms a heterodimer through electrostatic interactions between positively charged amino acid residues and negatively charged amino acid residues on the first and second charge polarized core domains.

53. The method of claim 51 or claim 52, wherein the first and/or second charge polarized core domain comprises a polypeptide linker, optionally selected from a flexible amino acid sequence, IgG hinge region, or antibody sequence.

54. The method of any one of claims 34 to 53, wherein the linker is a synthetic linker, optionally PEG.

55. The method of any one of claims 34 to 53, wherein the linker comprises the hinge-CH2-CH3 Fc domain derived from IgG1, optionally human IgG1.

56. The method of any one of claims 34 to 53, wherein the linker comprises the hinge-CH2-CH3 Fc domain derived from IgG4, optionally human IgG4.

57. The method of any one of claims 51 to 56, wherein the first and/or second charge polarized core domain further comprise peptides having positively and/or negatively charged amino acid residues at the amino and/or carboxy terminus of the charge polarized core domain.

58. The method of claim 57, wherein the positively charged amino acid residues include one or more of amino acids selected from His, Lys, and Arg.

59. The method of claim 57 or claim 58, wherein the positively charged amino acid residues are present in a peptide comprising positively charged amino acid residues in the first and/or the second charge polarized core domains.

60. The method of claim 59, wherein the peptide comprising positively charged amino acid residues comprises a sequence selected from YnXnYnXnYn (where X is a positively charged amino acid such as arginine, histidine or lysine and Y is a spacer amino acid such as serine or glycine, and where each n is independently an integer 0 to 4) (SEQ ID NO: 1), YYnXXnYYnXXnYYn (where X is a positively charged amino acid such as arginine, histidine or lysine and Y is a spacer amino acid such as serine or glycine, and where each n is independently an integer 0 to 4) (SEQ ID NO: 3), and YnXnCYnXnYn (where X is a positively charged amino acid such as arginine, histidine or lysine and Y is a spacer amino acid such as serine or glycine, and where each n is independently an integer 0 to 4) (SEQ ID NO: 5).

61. The method of claim 59 or claim 60, wherein the peptide comprising positively charged amino acid residues comprises the sequence RKGGKR (SEQ ID NO: 11) or GSGSRKGGKRGS (SEQ ID NO: 12).

62. The method of any one of claims 57 to 60, wherein the negatively charged amino acid residues may include one or more amino acids selected from Asp and Glu.

63. The method of claim 62, wherein the negatively charged amino acid residues are present in a peptide comprising negatively charged amino acid residues in the first and/or the second charge polarized core domains.

64. The method of claim 63, wherein the peptide comprising negatively charged amino acid residues comprises a sequence selected from YnZnYnZnYn (where Z is a negatively charged amino acid such as aspartic acid or glutamic acid and Y is a spacer amino acid such as serine or glycine, and where each n is independently an integer 0 to 4) (SEQ ID NO: 2), YYnZZnYYnZZnYYn (where Z is a negatively charged amino acid such as aspartic acid or glutamic acid and Y is a spacer amino acid such as serine or glycine, and where each n is independently an integer 0 to 4) (SEQ ID NO: 4), and YnZnCYnZnYn (where Z is a negatively charged amino acid such as aspartic acid or glutamic acid and Y is a spacer amino acid such as serine or glycine, and where each n is independently an integer 0 to 4) (SEQ ID NO: 6).

65. The method of claim 63 or claim 64, wherein the peptide comprising negatively charged amino acid residues comprises the sequence DEGGED (SEQ ID NO: 13) or GSGSDEGGEDGS (SEQ ID NO: 14).

66. The method of any one of claims 34 to 65, wherein the first domain and/or the heterodimeric protein modulates or is capable of modulating a γδ (gamma delta) T cell.

67. The method of claim 66, wherein the gamma delta T cell is selected from a cell expressing Vγ4, Vγ9δ2, or Vγ7δ4.

68. The method of claim 66 or claim 67, wherein the modulation of a gamma delta T cell is activation of a gamma delta T cell.

69. The method of any one of claims 34 to 68, wherein the heterodimeric protein is capable of forming a synapse between a gamma delta T cell and a tumor cell and/or the heterodimeric protein is capable of contemporaneous activation and targeting of gamma delta T cells to tumor cells.
